(19) United States
(12) Patent Application Publication
(10) Pub. No.: US 2007/0065837 A1
(43) Pub. Date: Mar. 22, 2007

(54) Methods and compositions for the detection of Chlamydia trachomatis

(75) Inventors: Meike Eickhoff, Ratzbek (DE); Thomas Grewing, Hamburg (DE)

Correspondence Address: SEED INTELLECTUAL PROPERTY LAW GROUP PLLC, 701 FIFTH AVE, SUITE 5400, SEATTLE, WA, 98104, US

(73) Assignee: QIAGEN Diagnostics GmbH, Hamburg (DE)

(21) Appl. No.: 11/233,505

(22) Filed: Sep. 21, 2005

(51) Int. Cl. C12Q 1/68; C07H 21/04

(52) U.S. Cl. 435/6; 536/24.1

(57) ABSTRACT

The present invention provides novel methods for determining the presence or absence of Chlamydia in a patient, as well as diagnostic kits useful in practicing the methods of the invention. The methods of the invention are based on nucleic acid amplification reactions to detect both Chlamydia genomic and cryptic plasmid sequences. In one embodiment, the methods involve using nucleic acid primers to specifically amplify the Chlamydia trachomatis ompA gene and cryptic plasmid. These methods provide both enhanced reliability and sensitivity of detection, thereby providing an accurate determination of the presence or absence of Chlamydia trachomatis in a patient.

### BACKGROUND OF THE INVENTION

1. Field of the Invention

The present invention relates to methods for determining the presence or absence of Chlamydia trachomatis in a patient, based upon the detection of genomic and cryptic plasmid sequences. The invention also provides diagnostic kits useful in practicing the methods of the invention.

2. Description of the Related Art

Bacteria of the species Chlamydia (C.) are of great epidemiological importance worldwide. Depending on transmission route and age of the patient, C. trachomatis causes infections of the eyes, lungs, or urogenital (urinary-genital) area. It is one of the most common causes of sexually transmitted diseases (STDs), although the majority of infected persons are not aware of it because Chlamydia infections are often asymptomatic. C. trachomatis infections may spread to the upper reproductive tract, including the uterus, fallopian tubes and ovaries. Scarring of the fallopian tubes may cause permanent damage to the reproductive system, resulting in infertility or life-threatening tubal pregnancy.

Historically, Chlamydia detection methods have included cell culture in the McCoy cell line, direct staining with the Papanicolaou cell line, and direct immunofluorescence and immunoassays, e.g., ELISAs. Cell culture was once regarded as the gold standard for C. trachomatis diagnosis. However, the sensitivity of cell culture compared with expanded standard tests is at best 75-80%, and probably nearer 55-65%. Serological detection methods are also available, but these are not species specific and, therefore, often produce meaningless results.

More current methods of diagnosing and detecting C. trachomatis, which are based on nucleic acid amplification techniques, allow routine diagnostic testing and do not require invasive sample collection. Examples of two nucleic acid amplification-based diagnostic assays include the Roche COBAS® AMPLICOR® C. trachomatis system and the Becton Dickinson Microbiology Systems BD Probe Tec ET. Both of these assays are based upon detecting the C. trachomatis cryptic plasmid.

The genome of the obligate intracellular bacterium C. trachomatis consists of a circular chromosome of 1.045 Mb and a conserved cryptic plasmid, which is approximately 7.5 kb in size and is present in multiple copies (5-10) in the organism. As used herein, the circular chromosome is referred to generally as “genomic sequence,” and the cryptic plasmid is referred to as “cryptic plasmid sequence.” The cryptic plasmid has practical importance as the favored target for nucleic acid amplification technologies, since the use of this multi-copy gene improves the possibility to detect infected patients. However, a few isolates of C. trachomatis have been described that do not contain the plasmid (Miyashita, N., et al., J. Infect Chemother. 7:113-116 (2001), Matsumoto, A., et al., J. Clin Microbiol. 36:3013-3019 (1998); Stothard, D. R., et al., Infect Immun. 66: 6010-6013 (1998), Farancena, A., et al., Infect Immun. 65: 2965-2969 (1997), and An, Q. and Olive, D. M., Mol Cell Probes. 8:429-435 (1994)). In addition, it was shown that the cryptic plasmid is not necessary for the survival and the replication of Chlamydia. Accordingly, it is thought that in the coming years, Chlamydia lacking the cryptic plasmid will increasingly appear, as most commercially available nucleic acid amplification-based detection systems are based on detection of the cryptic plasmid. This provides a selective advantage to Chlamydia lacking the cryptic plasmid, since they remain undetected, untreated, and, therefore, able to spread.

Clearly, there is a need in the art for an accurate, reliable, and easy-to-use diagnostic assay, capable of detecting all C. trachomatis, including those that do not contain the cryptic plasmid. The present invention meets this need by providing methods and reagents for detecting C. trachomatis based upon detection of C. trachomatis genome and cryptic plasmid nucleic acid sequences.

### BRIEF SUMMARY OF THE INVENTION

The present invention is drawn to compositions, methods, and kits for the detection of Chlamydia.

In one embodiment, the present invention provides a method for determining the presence or absence of Chlamydia in a patient, comprising: obtaining a biological sample from the patient; contacting at least a portion of the biological sample with a first oligonucleotide that hybridizes to a sequence of a Chlamydia genome under highly stringent conditions; contacting at least a portion of the biological sample with a second oligonucleotide that hybridizes to a sequence of a Chlamydia cryptic plasmid under highly stringent conditions; detecting in the sample amounts of a polynucleotide that hybridizes to either the first and second oligonucleotide; and comparing the amount of polynucleotide that hybridizes to either oligonucleotide to a control value, and therefrom determining the presence of Chlamydia in the patient. In one embodiment, the first oligonucleotide hybridizes to an ompA sequence. In another embodiment, the second oligonucleotide hybridizes to an open reading frame 8 sequence of the cryptic plasmid. In a particular embodiment, the first oligonucleotide hybridizes to an ompA sequence, and the second oligonucleotide hybridizes to an open reading frame 8 sequence of the cryptic plasmid. In one embodiment, said Chlamydia is Chlamydia trachomatis. In related embodiments, said control value is a predetermined cut-off value. In another embodiment, said control value is a negative control value determined using a third oligonucleotide that does not bind a Chlamydia sequence under high stringency conditions.

In a related embodiment, the present invention provides a plurality of oligonucleotides, comprising oligonucleotides primers that bind under high stringency conditions to C. trachomatis sequences. In one embodiment, two primers bind a genomic sequence, and two primers that bind a cryptic plasmid sequence. In one embodiment, the plurality of oligonucleotides further comprises a probe that binds to a C. trachomatis genomic sequence located between the binding sites of the two primers that bind a genomic sequence. In another embodiment, the plurality of oligonucleotides further comprises a probe that binds to a C. trachomatis cryptic plasmid sequence located between the binding sites of the two primers that bind a cryptic plasmid sequence. In particular embodiments, the primers that bind the genomic sequence are at a physically discrete location from the primers that bind the cryptic plasmid sequence.

In a further related embodiment, the present invention provides a diagnostic kit comprising: a first oligonucleotide that hybridizes to a sequence of a Chlamydia genome under highly stringent conditions; and a second oligonucleotide that hybridizes to a sequence of a Chlamydia cryptic plasmid under highly stringent conditions. In one embodiment, the first oligonucleotide hybridizes to an ompA sequence. In another embodiment, the second oligonucleotide hybridizes to an open reading frame 8 sequence of the cryptic plasmid. In a related embodiment, the first oligonucleotide hybridizes to an ompA sequence, and the second oligonucleotide hybridizes to an open reading frame 8 sequence of the cryptic plasmid. In one embodiment, said Chlamydia is Chlamydia trachomatis. In a related embodiment, the kit further comprises a positive control polynucleotide sequence that binds to either the first or second oligonucleotide under high stringency conditions. In another embodiment, the kit comprises primers that may be used to amplify an ompA sequence bound by the first oligonucleotide. In a related embodiment, the kit comprises primers that may be used to amplify an open reading frame 8 sequence of the cryptic plasmid that is bound by the second oligonucleotides. In yet another related embodiment, a kit further comprises a negative control polynucleotide sequence that does not bind to either the first or second oligonucleotides under high stringency conditions. Furthermore, in various embodiments, one or more of the oligonucleotides are fluorescently labeled.

### BRIEF DESCRIPTION OF THE SEVERAL VIEWS OF THE DRAWING(S)

FIGS. 1A and 1B provide graphical representations of real-time PCR results obtained using a DNA sample obtained from a patient infected with C. trachomatis. Fluorescence is indicated on the y-axis, and the number of amplification cycles is indicated on the x-axis. FIG. 1A shows results obtained using primers specific for ompA. FIG. 1B shows results obtained using primers specific for the cryptic plasmid.

### DETAILED DESCRIPTION OF THE INVENTION

The present invention provides methods and compositions for determining the presence or absence of Chlamydia in a biological sample, as well as methods of diagnosing and monitoring Chlamydia infection in the patient from whom the biological sample was derived, based upon the detection of Chlamydia polynucleotide sequences in the sample. In one embodiment, these methods are based upon the detection of a Chlamydia genomic sequence, such as, e.g., an ompA sequence. In particular embodiments, the present invention relates to the simultaneous or sequential evaluation or detection in a biological sample of at least two Chlamydia sequences, including one genomic sequence and one cryptic plasmid sequence. This simultaneous or sequential detection of two sequences by nucleic acid amplification methods is also referred to herein as “parallel amplification.” It has been found that by using such an approach, improved complementation and accuracy in detecting Chlamydia, including those that do not contain the cryptic plasmid, is achieved.

In another embodiment, the present invention provides oligonucleotides useful as amplification primers and assay probes, as well as sets and arrays of such oligonucleotides, and kits comprising the same, for the specific identification of Chlamydia, e.g., C. trachomatis, DNA or RNA in biological samples. These oligonucleotides, and sets thereof, are capable of detecting many or all C. trachomatis bacteria, including cryptic plasmid-free variants. In one embodiment, the oligonucleotides, or sets thereof, target the ompA gene of the genomic sequence and the cryptic plasmid sequence, without significantly cross-reacting with human DNA or other microorganism DNA or RNA.

A variety of definitions useful in understanding the present invention are provided herein. Unless indicated or defined otherwise, all scientific and technical terms used herein have the same meaning as commonly understood by those skilled in the relevant art. General definitions of many terms used herein are provided in: Dictionary of Microbiology and Molecular Biology, 2nd ed. (Singleton, et al., 1994, John Wiley & Sons, New York, N.Y.); The Harper Collins Dictionary of Biology (Hale & Marham, 1991, Harper Perennial, New York, N.Y.); and, Dorland's Illustrated Medical Dictionary, 27th ed. (W. A. Dorland, 1988, W.B. Saunders Co., Philadelphia, Pa.).

As used herein, the term “primer/probe specific for a DNA molecule” means an oligonucleotide sequence that has at least 80%, preferably at least 90% and more preferably at least 95%, identity to the DNA molecule in question. In certain embodiments, oligonucleotide primers and/or probes employed in the inventive diagnostic methods have at least about 10-40 nucleotides. In one embodiment, the oligonucleotide primers comprise at least 10 or at least 15 contiguous nucleotides of a Chlamydia genomic or cryptic plasmid sequence. DNA probes or primers comprising oligonucleotide sequences described above may be used alone or in combination with each other.

By “nucleotide sequence” or “nucleic acid sequence” is meant the sequence of nitrogenous bases along a linear information-containing molecule (e.g., DNA or RNA) that is capable of hydrogen-bonding with another linear information-containing molecule having a complementary base sequence. The terms are not meant to limit such information-containing molecules to polymers of nucleotides per se but are also meant to include molecular structures containing one or more nucleotide analogs or abasic subunits in the polymer. The polymers may include base subunits containing a sugar moiety or a substitute for the ribose or deoxyribose sugar moiety (e.g., 2′ halide- or methoxy-substituted pentose sugars), and may be linked by linkages other than phosphodiester bonds (e.g., phosphorothioate, methylphosphonate or peptide linkages).

By “oligonucleotide” is meant a polymeric chain of two or more chemical subunits, each subunit comprising a nucleotide base moiety, a sugar moiety, and a linking moiety that joins the subunits in a linear spacial configuration. An oligonucleotide may contain up to thousands of such subunits, but generally contains subunits in a range having a lower limit of between about 5 to about 10 subunits, and an upper limit of between about 20 to about 1,000 subunits. The most common nucleotide base moieties are guanine (G), adenine (A), cytosine (C), thymine (T) and uracil (U), although other rare or modified nucleotide bases able to form hydrogen bonds (e.g., inosine (I)) are well known to those skilled in the art. The most common sugar moieties are ribose and deoxyribose, although 2′-O-methyl ribose, halogenated sugars, and other modified and different sugars are well known. The linking group is usually a phosphorus-containing moiety, commonly a phosphodiester linkage, although other known phosphate-containing linkages (e.g., phosphorothioates or methylphosphonates) and non-phosphorus-containing linkages (e.g., peptide-like linkages found in “peptide nucleic acids” or PNAs) known in the art are included. Likewise, an oligonucleotide includes one in which at least one base moiety has been modified, for example, by the addition of propyne groups, so long as: (1) the modified base moiety retains the ability to form a non-covalent association with G, A, C, T or U; and, (2) an oligonucleotide comprising at least one modified nucleotide base moiety is not sterically prevented from hybridizing with a complementary single-stranded nucleic acid. An oligonucleotide's ability to hybridize with a complementary nucleic acid strand under particular conditions (e.g., temperature or salt concentration) is governed by the sequence of base moieties, as is well-known to those skilled in the art (Sambrook, J., et al., 1989, Molecular Cloning, A Laboratory Manual, 2nd ed. (Cold Spring Harbor Laboratory Press, Cold Spring Harbor, N.Y.), particularly pp. 7.37-7.57 and 11.47-11.57).

By “primer” or “amplification primer” is meant an oligonucleotide capable of binding to a region of a target nucleic acid or its complement and promoting, either directly or indirectly, nucleic acid amplification of the target nucleic acid. In most cases, a primer will have a free 3′ end that can be extended by a nucleic acid polymerase. Amplification primers include a base sequence capable of hybridizing via complementary base interactions to at least one strand of the target nucleic acid or a strand that is complementary to the target sequence. For example, in PCR, amplification primers anneal to opposite strands of a double-stranded target DNA that has been denatured. A thermostable DNA polymerase produces double-stranded DNA products, which are then denatured with heat, cooled and annealed to amplification primers, extends the primers. Multiple cycles of the foregoing steps (e.g., about 20 to about 50 thermic cycles) exponentially amplifies the double-stranded target DNA.

A “target-binding sequence” of an amplification primer is the portion that determines target specificity because that portion is capable of annealing to the target nucleic acid strand or its complementary strand. The complementary target sequence to which the target-binding sequence hybridizes is referred to as a primer-binding sequence. For primers or amplification methods that do not require additional functional sequences in the primer (e.g., PCR amplification), the primer sequence consists essentially of a target-binding sequence, whereas for other amplification methods (e.g., TMA or SDA), primers typically include additional specialized sequences adjacent to the target-binding sequence (e.g., an RNA polymerase promoter sequence adjacent to a target-binding sequence in a promoter-primer or a restriction endonuclease recognition sequence for an SDA primer).

By “target sequence” is meant the nucleotide base sequence of a nucleic acid strand, at least a portion of which is capable of being detected using a labeled oligonucleotide probe. Primers bind to a portion of a target sequence, which includes both complementary strands when the target sequence is a double-stranded nucleic acid.

A. Chlamydia DNA Sequences and Primers

Chlamydial sequences detected according to the methods provided herein include chlamydial genomic and/or cryptic plasmid sequences. These sequences include DNA and RNA sequences from any species of Chlamydia, including but not limited to C. trachomatis. They also include sequences from any or all Chlamydia serovars. The genomes of two Chlamydia species have been determined and made publicly available. These sequences are provided, e.g., as GenBank Accession Nos. M14738 and M19126, and GI:144550. Examples of other Chlamydia sequences are provided in Table 9, following the Examples section.

The C. trachomatis serovar D genome contains 1,042,519 nucleotides (nt) and estimated 894 protein coding genes (Stephens, R. S., et al., Science 282:754-759 (1998). C. trachomatis also contains an extrachromosomal cryptic plasmid genome of 7493 nt. Both genomes include a multigene family encoding sequence-variant putative outer membrane proteins, also referred to as polymorphic outer membrane proteins (POMPs), and the complete components for a type III secretion system. The Chlamydial outer membrane protein complex is composed primarily of three proteins; the major outer membrane protein (MOMP), and two cysteine-rich proteins, including the outer membrane complex B protein (OmcB) and the outer membrane complex A protein (OmcA). These proteins are encoded by the ompA, omcB, and omcA genes, respectively.

Any of these genomic sequences, as well as homologs, variants, and other alleles thereof, may be detected according to the methods of the present invention. In one embodiment, the methods of the present invention are used to detect an ompA gene. In particular embodiments, the ompA gene is the C. trachomatis ompA gene. This gene includes at least one region useful for the detection of many or all C. trachomatis serovars, which region corresponds to base pairs 470-601 of the sequence presented in GenBank Accession No. M14738, M19126 and GI:144550.

Chlamydial cryptic plasmids were first identified in C. trachomatis and the former C. psittaci(Lovett, M., et al., Plasmids of the genus Chlamydia. In: Current Chemotherapy and Infections Diseases, vol. 2, pp 1250-1252 (Eds. Nelson, J. and Grassi, C.) published by American Society of Microbiology, Washington, D.C. (1980). Various serovars, including C. trachomatis serovar B and C. trachomatis serovar L1, of the plasmid have been sequenced (Sriprakash, K. S. and Macavoy, E. S., Plasmid 18:205-214 (1987) and Hatt, C., et al., Nucleic Acids Res. 16:4053-4067 (1988)). Plasmid sequences of C. trachomatis were subsequently published for serovar L2 (Comanducci, M., et al., Molecular Microbiology 2:531-538 (1988)); serovar L1 (Thomas, N. S. and Clarke, I. N., Revised map of the Chlamydia trachomatis L1/440/LN plasmid. In: Proceedings of the 2nd Meeting of the European Society for Chlamydial Research, p. 42 (Eds. P-A Mardh et al.), published by Societa Editrice Esculapio, Bologna, Italy (1992)); and serovar D (Comanducci, M., et al., Plasmid 23:149-154 (1990)).

All plasmids from human C. trachomatis isolates are extremely similar, with less than 1% nucleotide sequence variation. All are about 7,500 nucleotides in size, with eight open reading frames computer-predicted to code for proteins of more than 100 amino acids, with short non-coding sequences between some of them only (Thomas, N. S., et al., Microbiology (UK) 143:1847-1854 (1997)). All chlamydial plasmids have four 22 base pair tandem repeats in the intergenic region between ORFs 1 and 8, plus AT rich clusters upstream of this region and an inverted repeat.

Some of the possible functions of these open reading frames are summarized in the table below, together with the relevant literature sources.

TABLE 1
Structural and functional properties of Chlamydia cryptic plasmid open
reading frames

| ORF | | |
|---|---|---|
| number | Function/Remarks | References |
| 1 | 305 amino acids in C. trachomatis L1 and C. muridarum Nigg | Tam et al., 1992 |
| | strain. 307 amino acids in avian C. psittaci N352. Major | Thomas & Clarke 1997 |
| | deletion, split ORF comprising only 185 amino acids in | |
| | equine C. pneumoniae pCpnE1. Plasmid encoded replication | |
| | protein thought to bind to 1 or more sets of tandem repeats. | |
| | Possible replication forks identified by electron microscopy. | |
| | Shares 32-35% amino acid identity with ORF 2. | |
| 2 | 330-335 amino acids in above strains. Similarity in | Buissan & Roy, 1991 |
| | properties to ORF1 suggests probably also involved in | Fahr et al., 1992 |
| | plasmid replication. ORF1 but not ORF2 has a deletion in the | Pearce 1990 |
| | equine C. pneumoniae strain, suggesting that ORF2 may be | Ricci et al., 1993 |
| | more important than ORF1 for plasmid replication. Unlike | Thomas & Clarke, 1994 |
| | other ORFs, transcribed from the complementary strand. | Thomas & Clarke, 1997 |
| | Contains conserved domains belonging to a family of | |
| | recombinase proteins. Activity probably regulated by short | |
| | antisense transcripts, which have been identified in vitro in C. muridarum | |
| | and the LGV biovar of C. trachomatis. | |
| 3 | Homologous with DnaB genes of E. coli and S. typhimurium | Hatt et al., 1988 |
| | & gene 12 of phage P22. DnaB is a helicase involved in | Sriprakash & Pearce, |
| | unwinding double stranded DNA during replication. An E. coli | 1990 |
| | promoter-like sequence has been found to occur upstream | Thomas & Clarke, 1997 |
| | from the plasmid-encoded dnaB gene. Sriprakash & Pearce | |
| | suggest such sequences may be evolutionary relics. | |
| 4 | 345-354 amino acids in the above strains. Function not | Thomas & Clarke, 1997 |
| | known. | |
| 5 | Encodes a 28 KDalton protein, pgp 3, which is known to be | Comanducci et al., 1993; |
| | produced in the chlamydial inclusion. Minor differences | 1994 |
| | between strains. Antibody to this protein can be detected in | Ratti et al., 1995 |
| | patient sera and also, for reasons that are unclear, in the sera | |
| | of HIV-infected patients. | |
| 6 | Predicted translation product is 101-102 amino acids in | Thomas & Clarke, 1997 |
| | above strains | |
| 7 | 243-261 amino acids in above strains. Shares partial | Thomas & Clarke, 1997 |
| | homology to several E. coli plasmid and phage encoded | |
| | proteins including SopA and ParA involved in regulation of | |
| | partitioning and copy number. | |
| 8 | Conserved. May function together with ORF 7 in similar | Ricci et al., 1995 |
| | manner to Sop A/B and ParA/B operons. | Comanducci et al., 1990 |

Any cryptic plasmid sequence may be identified according to the methods of the present invention. In one embodiment, the methods of the present invention detect sequences in ORF 8 of the cryptic plasmid.

Chlamydia genomic (i.e., chromosomal) and cryptic plasmid sequences may be detected using one or more primers/probes specific for a Chlamydia genomic or cryptic plasmid sequence or that bind a Chlamydia genomic or cryptic plasmid sequence under moderate or high stringency conditions. Hybridization techniques are well known in the art of molecular biology. For purposes of illustration, suitable moderate stringency conditions for testing the hybridization of a polynucleotide of this invention with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable high stringency hybridization conditions include those described above, with the exception that the temperature of hybridization is increased, e.g., to 60-65° C. or 65-70° C.

It will be appreciated by those skilled in the art that all of the primer and probe sequences of the present invention may be synthesized using standard in vitro synthetic methods. Also, it will be appreciated that those skilled in the art could modify primer sequences disclosed herein using routine methods to add additional specialized sequences (e.g., promoter or restriction endonuclease recognition sequences) to make primers suitable for use in a variety of amplification methods.

An oligonucleotide comprising a portion of any of the Chlamydia genomic or cryptic plasmid sequences described herein, or of a complementary sequence, may be used as a probe or primer to detect Chlamydia DNA or RNA sequences. In addition, variants of these sequences may be used. Such variants have at least 80%, at least 90%, at least 95%, or at least 99% sequence identity to a genomic or cryptic plasmid sequence described herein.

Preferably, the “percentage of sequence identity” is determined by comparing two optimally aligned sequences over a window of comparison of at least 20 positions, wherein the portion of the polynucleotide or amino acid sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity. One illustrative example of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information.

Two nucleotide or polypeptide sequences are said to be “identical” if the sequence of nucleotides or amino acid residues in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 12 contiguous positions, usually 20 to about 75, or 40 to about 50, in which a sequence may be compared to a reference sequence of the same number of contiguous positions after the two sequences are optimally aligned.

Optimal alignment of sequences for comparison may be conducted using the MegAlign program in the Lasergene suite of bioinformatics software (DNASTAR, Inc., Madison, Wis.), using default parameters. Alternatively, optimal alignment of sequences for comparison may be conducted by the local identity algorithm of Smith and Waterman (1981) Add. APL. Math 2:482, by the identity alignment algorithm of Needleman and Wunsch, J. Mol. Biol. 48:443 (1970), by the search for similarity methods of Pearson and Lipman, Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Examples of specific nucleotide sequences that may be used as probes or primers to detect ompA sequences are provided in Table 2.

TABLE 2
Exemplary Sequences for ompA-based Detection (Accession No.:
GI: 144550)

| Oligo | | | | Tm | Amplicon | |
|---|---|---|---|---|---|---|
| Name | Sequence (5′ → 3′) | Size | GC (%) | (° C.) | Size | Remark |
| FpCT | ATG AAA AAA CTC TTG AAA TCG GT | 23 | 30.4 | 55.0 | 106 | 474-496 bp |
| | (SEQ ID NO: 1) | | | | | |
| RpCT | CGA TCA TAA GGC TTG GTT CA | 20 | 45.0 | 55.0 | 106 | 560-579 bp |
| | (SEQ ID NO: 2) | | | | | |
| SChT1 | TTA GTI TTT GCC | 12 | 41.7 | 55.4 | 106 | 498-509 bp |
| | I: Inosin (SEQ ID NO: 3) | | | | | FAM-MGB |
| SChT2 | TTA GTI TTT GCC GCT TT | 17 | 41.2 | 66.3 | 106 | 498-514 bp |
| | I: Inosin (SEQ ID NO: 4) | | | | | FAM-MGB |

Examples of specific nucleotide sequences that may be used as probes or primers to detect cryptic plasmid sequences are provided in Table 3.

TABLE 3
Exemplary Sequences for cryptic plasmid-based Detection (Accession No.:
gi|4691224|emb|X06707.2|CTPLASCR)

| Oligo | | | GC | Tm | Amplicon | |
|---|---|---|---|---|---|---|
| Name | Sequence (5′ → 3′) | Size | (%) | (° C.) | Size | Remark |
| FpkP | AAA AGA GTT TCA ATC | 25 | 36.0 | 59.0 | 111 | 7113-7137 bp |
| | GAT CCC CTA T | | | | | |
| | (SEQ ID NO: 5) | | | | | |
| RpkP | GAG TTC GAC ATT CCA | 25 | 44.0 | 59.0 | 111 | 7223-7199 bp |
| | CAT ACT TTC C | | | | | |
| | (SEQ ID NO: 6) | | | | | |
| SkPa-B | AGG CGA TTT AAA AAC | 30 | 40.0 | 68.0 | 111 | 7167-7196 bp,, TaqMan- |
| | CAA GGT CGA TGT GAT | | | | | probe, 5′FAM, 3′BHQ1 |
| | (SEQ ID NO: 7) | | | | | |
| SkPr-B | TCA CAT CAT CGA CCT | 30 | 40.0 | 69.0 | 111 | 7192-7169 bp, Reverse |
| | TGG TTT TTA AAT CGC | | | | | TaqMan-probe, 5′FAM, |
| | (SEQ ID NO: 8) | | | | | 3′BHQ1 |

Probes and primers may be labeled with any of a variety of reporter groups, such as radionuclides, fluorogenic dyes, and enzymes. Each probe or primer may comprise zero, one, two, or more reporter groups. Accordingly, probes and primers may be, e.g., unlabeled, single-labeled, or dual-labeled. Labels may be added to the 5′ and/or 3′ nucleotides of an oligonucleotide, and/or to internal nucleotides.

Probes and primers may be modified to increase stability. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Probes and primers may be engineered for use in any of a variety of detection and amplification methods, including those described herein. Accordingly, probes and primers may include one or more specific features, e.g., labels or modifications, useful in performing a particular method of detection. In one embodiment, a probe or primer is designed for use in real-time PCR methods. A variety of fluorogenic probes may be used to perform various real-time PCR methods, including, but not limited to, the specific probes described herein.

The fluorogenic 5′ nuclease assay uses a dual-labeled fluorogenic oligonucleotide probe, called a TaqMan probe. TaqMan probes are typically between 20-24 bases long. The sequence of a TaqMan probe is complementary to an internal region of the target sequence amplified during PCR. The 3′-terminus is usually blocked, typically with PO₄, NH₂, or a blocked base, to prevent PCR priming from the internal TaqMan probe. The melting temperature (Tₘ) of the TaqMan probe is higher than that of the upstream PCR primer, to avoid a situation where the upstream primer is extended while the TaqMan probe has not yet annealed.

TaqMan probes are labeled with both a fluorescent reporter dye and a fluorescent quencher dye. Typically, TaqMan probes have a 5′-reporter dye, such as Fam, Tet, or Hex and a 3′-quencher group, such as Tamra or Dabcyl. Fluorescent dyes can be attached to oligonucleotides on the 3′-end, 5′-end, or to internal residues. A variety of dyes can be conjugated to oligonucleotides directly during synthesis using dye-phosphoramidites or dye-CPG derivatives. Other dyes can be conjugated to oligonucleotides post synthesis. A wide variety of suitable fluorophores are commercially available.

Fluorescence energy resonance transfer (FRET) occurs when energy passes from one fluorophore to another without emission of light. FRET requires that the two groups be in close proximity, usually 10-100 Å. In the intact TaqMan probe, energy is transferred via FRET from the short-wavelength fluorophore on one end to the long-wave fluorophore on the other end, quenching the short-wavelength fluorescence. During the PCR amplification reaction, the 5′ to 3′ exonuclease activity of the Taq polymerase cleaves the short-wavelength fluorophore from the bound TaqMan probe. Since the fluorophore is no longer in close proximity to the quencher, it begins to fluoresce. This fluorescence can be measured and is in direct proportion to the amount of target DNA.

The LightCycler assay utilizes two differently labeled probes that both bind to a region of target DNA between the PCR primers. The first oligonucleotide is labeled at the 3′-end (usually with fluorescing, and the second oligonucleotide is labeled at the 5′-end with a FRET acceptor (e.g., Cy5™ or TAMRA). The 3′-ends of both oligonucleotides are blocked to protect against chain elongation during PCR. The first oligonucleotide hybridizes to the target in such a way that its 3′-end is separated from the 5′-end of the second oligonucleotide by no more than 1 base. When no complementary sequence is present, only the fluorescence of the donor is visible. If the target is present, the labeled probes will hybridize with the target and FRET can occur. The fluorescence intensity is proportional to the amount of PCR product during the exponential phase of PCR.

Minor groove binding molecules are utilized during quantitative PCR in both target sequence specific and non-specific methods. The addition of minor groove binding molecules to fluorogenic 5′ nuclease probes increases Tₘ, thus allowing for the use of shorter probes. In addition, the presence of the minor groove binder stabilizes A-T bonds more than G-C bonds, thus reducing the influence of target sequence on Tₘ.

Eclipse probes incorporate a novel crescent-shaped molecule (MGB) that binds to the minor groove of a DNA helix, resulting in a greater than 15-fold increase in Tₘ. Eclipse probes contain both the minor groove binder and a proprietary quencher (Eclipse quencher) at the 5′ end of the oligonucleotide, and a fluorescent dye (usually FAM) at the 3′ end.

Molecular beacons are dual-labeled oligonucleotides having a fluorescent reporter group at one end and a fluorescence quencher group at the other end. Molecular beacons are further designed such that in the absence of target, the molecules form an internal hairpin that brings the reporter and quencher groups in physical proximity, resulting in efficient quenching of the reporter. In the presence of target, the probe molecules unfold and hybridizes; reporter and quencher are now physically separated, and the reporter dye will emit fluorescence signal upon stimulation.

The Cycling Probe method of signal amplification for the detection of a nucleic acid target functions by allowing a single target molecule to act as a catalyst in converting a large number of probe molecules to a unique, detectable form. The catalytic amplification process is the “cycling probe reaction.” The basis of the system is an oligomer probe construction consisting of a DNA-RNA-DNA sequence.

The invention further provides sets of oligonucleotides primers/probes that may be used to detect one or more Chlamydia sequences. Such sets of oligonucleotides may include one or more oligonucleotides specific for each Chlamydia sequence being detected. In one embodiment, a set of oligonucleotides includes two oligonucleotides capable of amplifying a first Chlamydia sequence, e.g., ompA, by PCR, and two oligonucleotides capable of amplifying a second Chlamydia sequence, e.g., cryptic plasmid, by PCR. Sets of sequences may further include one or more oligonucleotides specific for a positive control or negative control DNA sequence. In addition, sets of oligonucleotides may include one or more internal primers/probes, which bind to a region of an amplicon, thereby facilitating detection. Sets of oligonucleotides may be provided in one or more tubes or containers. In one embodiment, primers specific for each target sequence are provided in separate tubes or containers, or are provided in discrete regions of an array, such as, e.g., different wells of a microtitre plate.

B. Amplification and Detection Methods

The methods of the present invention are directed to detecting or determining the presence or absence of Chlamydia in a biological sample. Accordingly, the methods of the invention may be practiced for a variety of purposes, including, e.g., to detect or diagnose Chlamydia infection in a patient, to monitor the progression of Chlamydia infection, and to assess the effect of treatment on Chlamydia infection of a patient. Thus, it is understood that the methods described herein may be routinely adapted for any purpose, e.g., by practicing the method in samples obtained from a patient at two or more different times and comparing the amount of Chlamydia detected in the different samples. While any species of Chlamydia may be detected, in particular embodiments, the methods of the invention are directed to the detection of C. trachomatis.

The methods of the present invention typically involve: isolating DNA from a biological sample obtained from a patient and detecting an amount of Chlamydia sequence is the isolated DNA using one or more oligonucleotides specific for a Chlamydia sequence, e.g., genomic sequence, thereby determining the presence or absence of Chlamydia in the biological sample. In one embodiment, the methods of the invention detect two or more Chlamydia sequences, including both genomic and cryptic plasmid sequences. In a particular embodiment, the genomic sequence is ompA and/or the cryptic plasmid sequence is a region of ORF8.

The methods of the present invention are routinely practiced using a biological sample obtained from a patient. The biological sample may be any type of biological sample suspected of or diagnosed as containing Chlamydia or Chlamydia DNA sequences. In one embodiment, for example, the biological sample is a tissue sample suspected of containing Chlamydia-infected cells. In other embodiments, the biological sample is selected from the group consisting of a biopsy sample, lavage sample, sputum sample, serum sample, peripheral blood sample, lymph node sample, bone marrow sample, urine sample, semen sample, and pleural effusion sample. In further embodiments, the biological sample is obtained by swabbing the eye, cervix or urogenital area. Swabs obtained from these regions include, e.g., eye, endocervical and urethral swabs. Biological samples should preferentially be obtained and stored under conditions established in the art to preserve the integrity of DNA. Typically, samples may be stored at 2-8° C. for seven days before being tested.

The methods of the present invention may be used in amplification and detection methods that use nucleic acid substrates, i.e., DNA or RNA, isolated from biological samples by any of a variety of well-known and established methodologies (e.g., Sambrook, J., et al., 1989, Molecular Cloning, A laboratory Manual, 2^{nd} ed. (Cold Spring Harbor Laboratory Press, Cold Spring Harbor, N.Y.), pp. 7.37-7.57; Lin, L., et al., 1993, “Simple and Rapid Sample Preparation Methods for Whole Blood and Blood Plasma” in Diagnostic Molecular Microbiology, Principles and Applications (Persing, D., Eds., American Society for Microbiology, Washington, D.C.), pp. 605-616).

A variety of commercial kits are available for isolating DNA from a biological sample, and any of these may be used. Examples of such kits include, but are not limited to, the PureArt™ Viral RNA Mini Kit (Qiagen Diagnostics, Hamburg, Germany), the QIAamp™ Viral RNA Mini Kit (QIAGEN, Germany), the PureArt™ DNA Mini Kit (Qiagen Diagnostics, Hamburg, Germany), the QIAamp™ DNA Mini Kit (QIAGEN, Germany) and the MagNA Pure™ DNA Isolation Kit (Roche Diagnostics, Basel, Switzerland).

In certain embodiments, carrier RNA is used during the DNA or RNA isolation procedure to increase extraction efficiency and DNA/RNA yield. Carrier RNA may be supplied in a commercial kit or it may be obtained independent and used in concert with a commercial kit. In one embodiment, the carrier RNA is RNA-Homopolymer Poly(A) (GE Healthcare, Germany), which is recommended for the extraction of nucleic acids from cell free body fluids and material low in DNA/RNA content.

An internal or positive control DNA may be used in practicing the methods of the invention. In certain embodiments, the internal control DNA is used to verify success of DNA isolation from the biological sample and/or to check for inhibitors of nucleic acid amplification. In various embodiments, control DNA may be added to a biological sample before the DNA purification process, during the purification process (e.g., added to lysis buffer containing the sample), or before the amplification process. Internal control DNA may be any DNA sequence that would yield a positive result given the assay performed. In certain embodiments, the internal control DNA is a C. trachomatis genomic, e.g., ompA, or cryptic plasmid sequence.

A negative control DNA sample may be used in practicing the methods of the invention. A negative control DNA sample does not contain sequences targeted for detection, i.e., is not bound by the probes/primers used for detection. A negative control DNA may be included to verify that reagents used in practicing the detection method are not contaminated with Chlamydia sequences.

In certain embodiments of the invention, the step of detecting Chlamydia comprises detecting Chlamydia DNA or RNA sequences in a biological sample, for example, using a nucleic acid hybridization technique or a nucleic acid amplification method. Such methods for detecting nucleic acid sequences are well-known and established in the art and may include, but are not limited to, Southern and Northern blot analysis, nuclear run-off assays, primer extension assays, S1 nuclease assays, transcription-mediated amplification (TMA), polymerase chain reaction amplification (PCR), ligase chain reaction amplification (LCR), strand displacement amplification (SDA), and nucleic acid sequence based amplification (NASBA), as known in the art and further described herein.

By “amplification” or “nucleic acid amplification” is meant production of multiple copies of a target nucleic acid that contains at least a portion of the intended specific target nucleic acid sequence (e.g., genomic or cryptic plasmid sequence). The multiple copies may be referred to as amplicons or amplification products. In certain embodiments, the amplified target contains less than the complete target gene sequence (introns and exons) or an expressed target gene sequence (spliced transcript of exons and flanking untranslated sequences). For example, specific amplicons may be produced by amplifying a portion of the target polynucleotide by using amplification primers that hybridize to, and initiate polymerization from, internal positions of the target polynucleotide. Preferably, the amplified portion contains a detectable target sequence that may be detected using any of a variety of well-known methods.

In particular embodiments, amplification is performed using the polymerase chain reaction (PCR). Many well-known methods of nucleic acid amplification require thermocycling to alternately denature double-stranded nucleic acids and hybridize primers; however, other well-known methods of nucleic acid amplification are isothermal. PCR (U.S. Pat. Nos. 4,683,195; 4,683,202; 4,800,159; 4,965,188), uses multiple cycles of denaturation, annealing of primer pairs to opposite strands of a target sequence, and primer extension to exponentially increase copy numbers of the target sequence. In a variation called RT-PCR, reverse transcriptase (RT) is used to make a complementary DNA (cDNA) from mRNA, and the cDNA is then amplified by PCR to produce multiple copies of DNA. The ligase chain reaction (Weiss, R., Science 254: 1292 (1991)), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Since PCR was initially described in the 1980's, numerous technical improvements have been made in PCR product detection. Many of these methodologies are based upon the inherent 5′ exonuclease activity of the Taq DNA polymerase used in the PCR reaction. The first reported 5′ nuclease assay was a PCR reaction conducted in the presence of a ³²P-labeled oligonucleotide complementary to a region of target DNA between the two PCR oligonucleotides. During PCR, the 5′ nuclease activity of Taq degraded the bound labeled oligonucleotide, releasing free probe in an amount corresponding to the amount of target sequence within a sample (Holland et al., Proc. Natl. Acad. Sci. USA 88:7276-7280 (1991)). Subsequent improvements and variations on the original 5′ nuclease assay utilize a variety of different probes, different numbers of internal binding oligonucleotides, and different techniques to optimize sensitivity. Several widely used techniques include fluorogenic 5′ nuclease assays utilizing a single dual-labeled probe (i.e., TaqMan assay) or dual single-labeled probes (i.e., LightCycler assay). In addition, specialized fluorogenic probes (e.g., Molecular Beacons) and probes incorporating minor groove binders have been developed.

Real-time PCR systems are based on the detection and quantitation of a signal emitted from a fluorescent reporter. This signal increases in direct proportion to the amount of PCR product in a reaction. By recording the amount of fluorescence emission at each cycle of the amplification process, it is possible to monitor the PCR reaction during exponential phase where the first significant increase in the amount of PCR product correlates to the initial amount of target template. The higher the starting copy number of the nucleic acid target, the sooner a significant increase in fluorescence is observed. In one embodiment, a significant increase in fluorescence above the baseline value measured during the 3-15 cycles indicates the detection of accumulated PCR product. Real-time PCR may be practiced using a variety of different labelled primers, including but not limited to those described here, e.g., Taqman, LightCycler, etc.

Another method is strand displacement amplification (Walker, G., et al., 1992, Proc. Natl. Acad. Sci. USA 89:392-396; U.S. Pat. Nos. 5,270,184 and 5,455,166), commonly referred to as SDA, which uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (European Pat. No. 0 684 315).

Other amplification methods include: nucleic acid sequence based amplification (U.S. Pat. No. 5,130,238), commonly referred to as NASBA; one that uses an RNA replicase to amplify the probe molecule itself (Lizardi, P., et al., 1988, BioTechnol. 6: 1197-1202), commonly referred to as Qβ replicase; a transcription based amplification method (Kwoh, D., et al., 1989, Proc. Natl. Acad. Sci. USA 86:1173-1177); self-sustained sequence replication (Guatelli, J., et al., 1990, Proc. Natl. Acad. Sci. USA 87:1874-1878); and, transcription mediated amplification (U.S. Pat. Nos. 5,480,784 and 5,399,491), commonly referred to as TMA. For further discussion of known amplification methods see Persing, David H., 1993, “In Vitro Nucleic Acid Amplification Techniques” in Diagnostic Medical Microbiology: Principles and Applications (Persing et al., Eds.), pp. 51-87 (American Society for Microbiology, Washington, D.C.).

TMA employs an RNA polymerase to produce multiple RNA transcripts of a target region (U.S. Pat. Nos. 5,480,784 and 5,399,491). TMA uses a “promoter-primer” that hybridizes to a target nucleic acid in the presence of a reverse transcriptase and an RNA polymerase to form a double-stranded promoter from which the RNA polymerase produces RNA transcripts. These transcripts can become templates for further rounds of TMA in the presence of a second primer capable of hybridizing to the RNA transcripts. Unlike PCR, LCR or other methods that require heat denaturation, TMA is an isothermal method that uses an RNase H activity to digest the RNA strand of an RNA:DNA hybrid, thereby making the DNA strand available for hybridization with a primer or promoter-primer. Generally, the RNase H activity associated with the reverse transcriptase provided for amplification is used.

In an illustrative TMA method, one amplification primer is an oligonucleotide promoter-primer that that comprises a promoter sequence which becomes functional when double-stranded, located 5′ of a target-binding sequence, which is capable of hybridizing to a binding site of a target RNA at a location 3′ to the sequence to be amplified. A promoter-primer may be referred to as a “T7-primer” when it is specific for T7 RNA polymerase recognition. Under certain circumstances, the 3′ end of a promoter-primer, or a subpopulation of such promoter-primers, may be modified to block or reduce primer extension. From an unmodified promoter-primer, reverse transcriptase creates a cDNA copy of the target RNA, while RNase H activity degrades the target RNA. A second amplification primer then binds to the cDNA. This primer may be referred to as a “non-T7 primer” to distinguish it from a “T7-primer”. From this second amplification primer, reverse transcriptase creates another DNA strand, resulting in a double-stranded DNA with a functional promoter at one end. When double-stranded, the promoter sequence is capable of binding an RNA polymerase to begin transcription of the target sequence to which the promoter-primer is hybridized. An RNA polymerase uses this promoter sequence to produce multiple RNA transcripts (i.e., amplicons), generally about 100 to 1,000 copies. Each newly-synthesized amplicon can anneal with the second amplification primer. Reverse transcriptase can then create a DNA copy, while the RNase H activity degrades the RNA of this RNA:DNA duplex. The promoter-primer can then bind to the newly synthesized DNA, allowing the reverse transcriptase to create a double-stranded DNA, from which the RNA polymerase produces multiple amplicons. Thus, a billion-fold isothermic amplification can be achieved using two amplification primers.

While the methods described above are provided for exemplary purposes, it is understood that any amplification method known in the art may be used to practice the methods of the present invention.

Any of a variety of methods may be utilized for determining the presence or absence of (i.e., “detecting”) an amplified nucleic acid, such as, for example, hybridizing a labeled probe to a portion of the amplified product. A labeled probe is an oligonucleotide that specifically binds to another sequence and contains a detectable group that may be, for example, a fluorescent moiety, chemiluminescent moiety, radioisotope, biotin, avidin, enzyme, enzyme substrate, or other reactive group. In one embodiment, a labeled probe includes an acridinium ester (AE) moiety that can be detected chemiluminescently under appropriate conditions (as described, e.g., in U.S. Pat. No. 5,283,174). In other embodiments, labeled probed include a fluorogenic dye.

In particular embodiments, the amount of amplified product is measured using a fluorescent label, e.g., fluorogenic dye, attached to one or more of the primers used to amplify a target Chlamydia sequence or to a probe that binds a sequence of an amplified product (amplicon).

In certain embodiments, the amount of amplified product is measured using a fluorescent dye, such as SYBR Green I. SYBR Green I binds to the minor groove of the DNA double helix. In solution, the unbound dye exhibits very little fluorescence. However, fluorescence is greatly enhanced upon DNA binding. When PCR is conducted in the presence of SYBR Green I, the dye molecules bind to newly synthesized DNA, resulting in a dramatic increase in detectable fluorescence. Upon denaturation of the DNA for the next PCR cycle, the dye molecules are released and the fluorescence signal falls. Fluorescence is measured at the end of each elongation step of every PCR cycle to monitor the increasing amount of amplified DNA. Thus, SYBR Green I allows for specific sequence identification and quantification.

Other well-known detection techniques include, for example, gel filtration, gel electrophoresis and visualization of the amplicons, and High Performance Liquid Chromatography (HPLC). The detecting step may either be qualitative or quantitative, although quantitative detection of amplicons may be preferred, as the amount of Chlamydia sequences may be indicative of the degree of infection, progression of infection and/or responsiveness to therapy.

In one embodiment, the methods of the invention are used to detect Chlamydia, based upon the detection or amplification of one sequence, e.g., a Chlamydia genomic sequence, such as ompA. However, in particular embodiments, the methods of the invention include the amplification or detection of two or more Chlamydia sequences, e.g., an ompA sequence and a cryptic plasmid sequence. In addition, the methods o the invention may further include the amplification or detection of one or more additional sequences, including, e.g., a positive control or negative control sequence. Detection or amplification of two or more sequences may be performed sequentially or simultaneously. In addition, detection or amplification of two or more sequences may be performed in a single amplification or detection procedures, or in separate amplification or detection procedures. For example, an amplification procedure may be performed in a single reaction mixture, using probes/primers specific for two or more sequences, wherein said probes/primers are differentially labeled, such that the amount of each amplified target sequence may be determined, based upon detecting the amount of label specific to each sequence. In another example, amplification of each target sequence is performed is separate reaction mixtures, but the amplification process is performed on both at the same time. In one embodiment, the separate reactions are performed in different wells of a microtitre plate or other array, and amplification is performed on the entire plate at once.

The above descriptions are intended to be exemplary only. It will be recognized that numerous other assays exist that can be used for amplifying and/or detecting DNA sequences in biological samples. Such methods are also considered within the scope of the present invention.

The presence or absence of Chlamydia in a biological sample may be determined based upon the amount of Chlamydia sequence determined by the detection methods. In one embodiment, the detection of any amount of Chlamydia sequence indicates the presence of Chlamydia in a biological sample, while the detection of no amount of Chlamydia sequence indicates the absence of Chlamydia in a sample. In other embodiments, the presence or absence of Chlamydia in a sample is determined by comparing the amount of one or more detected Chlamydia sequences to a control amount or a predetermined cut-off value.

In various embodiments, a positive control amount is an amount detected when detection is performed using a positive control sequence. A negative control amount is an amount detected when detection is performed using a negative control sequence or a probe/primers that do not specifically bind a Chlamydia sequence. In one embodiment, the presence of Chlamydia in a sample is indicated by an amount detected that is at least two, three, or five times greater than the negative control amount. In a related embodiment, the presence of Chlamydia in a sample is indicated by an amount that is at least 10%, at least 25%, at least 50%, or at least 100% of the positive control amount. In one embodiment, the absence of Chlamydia in a sample is indicated by an amount detected that is less than two times greater than the negative control amount. In another embodiment, the absence of Chlamydia in a sample is indicated by an amount detected that is less than 10% or less than 25% of the positive control amount. The control amounts may be determined before, during, or in parallel to determining the amount of Chlamydia sequences in a sample, e.g., using the same reagents and procedures.

A predetermined cut-off value used in the methods described herein for determining the presence of C. trachomatis can be readily identified using well-known techniques. For example, in one illustrative embodiment, the predetermined cut-off value for the detection of Chlamydia is the average mean signal obtained when the relevant method of the invention is performed on suitable negative control samples, e.g., samples from patients without Chlamydia. In another illustrative embodiment, a sample generating a signal that is two or three standard deviations above the predetermined cut-off value is considered positive.

Amounts of detected Chlamydia sequence (or other sequence) may be measured using routine procedures, depending upon the particular detection mean employed. For example, when using a fluorescently labeled probe/primer to detect a sequence, a fluorometer is used to measure the amount of fluorescence, which, in certain embodiments, correlates to the amount of amplified sequence. In one embodiment, a LightCycler® instrument is used. This may be, e.g., a LightCycler® 1.1/1.2/1.5 or 2.0 instrument and, in some embodiments, commercially available software and/or data analytical tools are used to analyze the results.

C. Diagnostic Kits

The present invention also includes kits useful for practicing the methods of the invention. Kits comprise reagents for practicing the methods of the invention, and may also include instructions for practicing methods of the invention. Kits may include one or more vials comprising various reagents useful in practicing the methods of the invention.

Kits of the present invention may include a variety of different oligonucleotides capable of binding a Chlamydia polynucleotide sequence under high stringency conditions. For example, a kit may include either or both of: oligonucleotides probes useful in detecting amplified Chlamydia sequences; and oligonucleotides primers capable of amplifying Chlamydia sequences. Thus, in one embodiment, a kit comprises an oligonucleotides probe that binds under high stringency conditions to a Chlamydia genomic sequence and, optionally, an oligonucleotides probe that binds under high stringency conditions to a Chlamydia cryptic plasmid sequence. In another exemplary embodiment, a kit comprises one or two (or more) oligonucleotides primers that bind a Chlamydia genomic sequence under high stringency conditions, and which are capable of amplifying a Chlamydia genomic sequence. Such kits may further include one or two (or more) oligonucleotides primers that bind a Chlamydia cryptic plasmid sequence under high stringency conditions, and which are capable of amplifying a Chlamydia cryptic plasmid sequence. In addition, in certain embodiments, kits comprise both oligonucleotide primers for amplification of Chlamydia sequences, as well as oligonucleotide probes for detecting amplified Chlamydia sequences.

In various embodiments, a kit includes one or more oligonucleotides that bind under high stringency conditions to a Chlamydia genomic sequence. Such kits may optionally include one or more oligonucleotides that bind under high stringency conditions to a Chlamydia cryptic plasmid sequence.

In certain embodiment, kits comprise one or more oligonucleotides capable of binding to a Chlamydia genomic sequence under high stringency conditions. In one embodiment, a kit comprises two oligonucleotide probes, including one that binds to a Chlamydia genomic sequence and one that binds to a cryptic plasmid sequence, respectively, under high stringency conditions.

In certain embodiments, kits comprise one or more oligonucleotides that bind to a Chlamydia genomic sequence at high stringency. In one embodiment, a kit comprises two oligonucleotides primers that bind to a Chlamydia genomic sequence at high stringency, and which may be used to perform PCR amplification of a Chlamydia genomic sequence. In particular embodiments, kits further include one or more oligonucleotides that bind to a Chlamydia cryptic plasmid sequence under high stringency conditions. In certain embodiments, kits comprise two oligonucleotides that bind to a Chlamydia cryptic plasmid sequence at high stringency, and which may be used to perform PCR amplification of a Chlamydia cryptic plasmid sequence. Any of the oligonucleotides described herein may be included in the kits. One or more of the oligonucleotides included in the kits may be labeled, e.g., fluorescently labeled. In certain embodiments, oligonucleotides that bind to genomic sequences are labeled with a different label than the label used to label oligonucleotides that bind cryptic plasmid sequences, thereby enabling specific detection of amplified genomic and/or cryptic plasmid sequences. In other embodiments, oligonucleotides that bind to genomic sequences and oligonucleotides that bind to cryptic plasmid sequences are labeled with the same label, thereby allowing detection of any amplified Chlamydia sequence by means of detecting a single fluorescent signal. In certain embodiments, oligonucleotides that bind to genomic sequence are provided in a different vial than oligonucleotides that bind to cryptic plasmid sequences, to allow selective use of different oligonucleotides, although in other embodiments, they are provided in the same vial.

In one particular embodiment, a kit comprises the components detailed in Table 4. The colors indicate different vials included in the kit.

TABLE 4
Exemplary kit components

| | Labelling | Art. No. 2200-01 | Art. No. 2200-02 | Art. No. 2200-03 |
|---|---|---|---|---|
| | and contents | 24 reactions | 48 reactions | 96 reactions |
| Blue | C. trachomatis Plus | 2 × 12 rxns | 4 × 12 rxns | 8 × 12 rxns |
| | LC Master | | | |
| Yellow | C. trachomatis Plus | 1 × 400 μl | 1 × 400 μl | 1 × 400 μl |
| | LC Mg-Sol | | | |
| Red | C. trachomatis Plus LC | 1 × 200 μl | 1 × 200 μl | 1 × 200 μl |
| | Positive Control | | | |
| | (1 × 10⁴ cop/μl) | | | |
| Green | C. trachomatis Plus LC | 1 × 1000 μl | 1 × 1000 μl | 2 × 1000 μl |
| | IC | | | |
| White | Water (PCR grade) | 1 × 1000 μl | 1 × 1000 μl | 1 × 1000 μl |

IC = Internal Control

Kits may further comprise other reagents. For example, in certain embodiments, kits comprise one or more of nucleotides, buffers, water, and enzymes, e.g., Taq polymerase, useful in performing nucleic acid amplification assays. In certain embodiments, kits comprise positive or negative control DNA. In one embodiment, positive control DNA is provided at a concentration of approximately 1×10⁴ copies/μl.

In one specific embodiment, a kit comprises: (1) three oligonucleotides that bind to a C. trachomatis ompA sequence under high stringency conditions and may be used to amplify and/or detect a ompA sequence; and (2) four oligonucleotides that bind to a C. trachomatis cryptic plasmid sequence under high stringency conditions and may be used to amplify and/or detect a cryptic plasmid sequence. In addition, the kit contains a heterologous amplification system to identify possible PCR inhibition. This is detected as an Internal Control (IC). These sets of oligonucleotides may be provided in the same or two or more different vials.

In another specific embodiment, a kit comprises: (1) a first vial comprising three oligonucleotides that bind to a C. trachomatis ompA sequence under high stringency conditions and may be used to amplify and/or detect an ompA sequence, four oligonucleotides that bind to a C. trachomatis cryptic plasmid under high stringency conditions and may be used to amplify and/or detect a cryptic plasmid sequence, and nucleotides, in a master mix; (2) a second vial comprising a magnesium solution; (3) a third vial comprising a positive control DNA; and (4) a fourth vial comprising a internal control DNA.

### EXAMPLES

### Example 1

### PCR Detection of C. trachomatis Using Primers Specific for ompA

To demonstrate that detection of genomic sequences is a reliable means of diagnosing C. trachomatis infection of a patient when the cryptic plasmid is not present, samples were obtained from various patients, and DNA was extracted and amplified by PCR using primers specific for ompA or the cryptic plasmid. DNA of 176 patient samples was extracted using the QIAamp DNA Mini Kit (QIAGEN). Primers and probes used for amplification and detection are described in Tables 2 and 3. For amplification and detection, the RealArt™ C. trachomatis Plus RG PCR Kit was used on the Rotor-Gene™ 3000 instrument (Corbett Research).

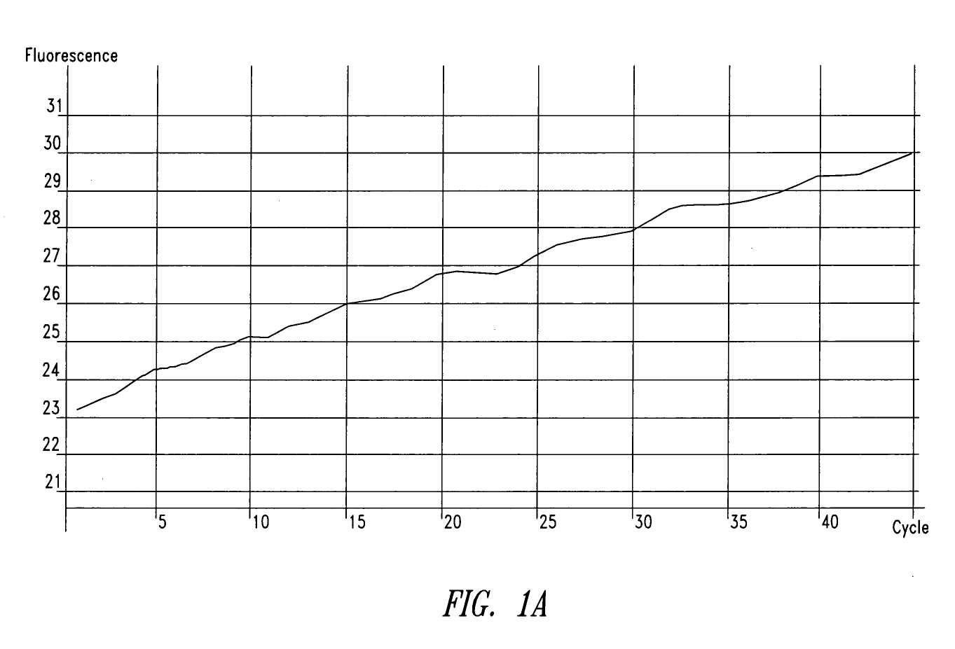

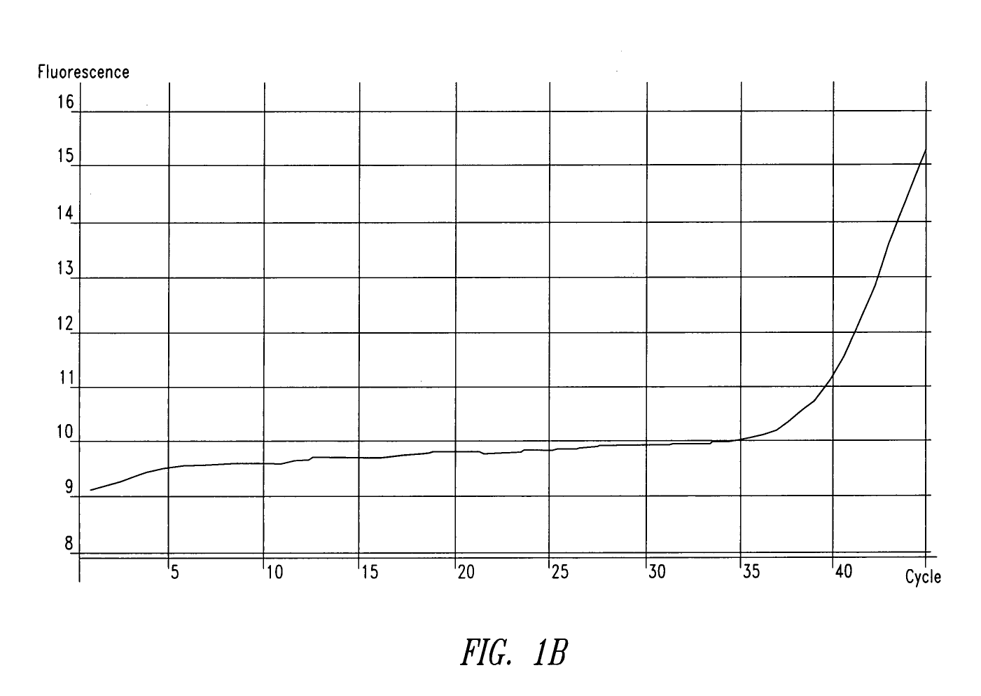

In one patient sample, C. trachomatis was repeatedly detected using the ompA-specific primers, but it was not detected using primers specific for the cryptic plasmid, as shown in FIG. 1. The cryptic plasmid-based PCR reaction is approximately 10-fold more sensitive compared to the ompA-based assay. Therefore, the negative result is not due to lower sensitivity.

These results demonstrate that detection of the cryptic plasmid, alone, is not sufficient to detect C. trachomatis infection in all patients, and indicates that diagnostic assays based upon the detection of other C. trachomatis polynucleotide sequences, such as ompA, can reliably detect C. trachomatis infection.

### Example 2

### Analytical Sensitivity of Parallel Amplification Detection of C. Trachomatis Using Primers Specific for ompA and the Cryptic Plasmid

The analytical detection limit, as well as the analytical detection limit in consideration of the purification method (sensitivity limits), of the parallel amplification methods, were assessed. The analytical detection limit in consideration of the purification was determined using C. trachomatis-positive clinical samples in combination with a particular extraction method. In contrast, the analytical detection limit was determined without clinical specimens and independent from the selected extraction methods, using serovars of known concentration.

To determine the analytical sensitivity, a C. trachomatis serovar dilution series was set up from 0.66 to nominal 0.002 C. trachomatis copy equivalents/μl and analyzed on the LightCycler® 1.1/1.2/1 instrument via parallel amplification using primers specific for C. trachomatis genomic and cryptic plasmid sequences. Testing was carried out on three different days on eight replicates (n=24). The results were determined by probit analysis. The analytical detection limit was consistently 0.1 copies/μl (p=0.05), indicating that there was a 95% probability that 0.1 copies were detected.

The analytical sensitivity in consideration of various purification methods was assessed using a dilution series of a C. trachomatis serovar from 2.1 to nominal 0.00066 C. trachomatis copy equivalents/μl spiked in clinical swab specimens. These were subjected to DNA extraction using either the QIAGEN QIAamp DNA Mini Kit (extraction volume: 200 μl; elution volume: 100 μl) or the Roche Diagnostics MagNA Pure DNA Isolation Kit (extraction volume: 100 μl; elution volume: 100 μl). Each of eight dilutions for each extraction methods were analyzed on the LightCycler® 1.1/1.2/1 instrument via parallel amplification using primers specific for C. trachomatis genomic and cryptic plasmid sequences. Testing was carried out on three different days on eight replicates. The results were determined by probit analysis. The analytical detection limit in consideration of QIAamp DNA Mini Kit purification was consistently 0.4 copies/μl (p=0.05), indicating that there was a 95% probability that 0.4 copies/μl were detected. The analytical detection limit in consideration of MagNA Pure DNA Isolation Kit purification was consistently 0.5 copies/μl (p=0.05), indicating that there was a 95% probability that 0.5 copies/μl were detected. Similar results were obtained using a LightCycler® 2.0 instrument.

### Example 3

### Specificity and Robustness of Parallel Amplification Detection of C. trachomatis Using Primers Specific for C. trachomatis ompA and Cryptic Plasmid

The specificity of the parallel amplification methods was assessed by analyzing 100 different C. trachomatis negative swabs, 30 C. trachomatis negative urine samples, and 30 C. trachomatis negative semen samples using primers specific for C. trachomatis ompA and cryptic plasmid sequences, as provided in Tables 2 and 3. None of these assays generated a positive signal. In addition, C. trachomatis specificity was validated by testing samples positive for other pathogens for cross-reactivity. As shown in Table 5, none of the tested pathogens generated a positive signal. Positive controls generated a signal. These results indicate that the methods of the invention are specific for C. trachomatis, and do not produce false positive results.

TABLE 5
C. trachomatis specificity of ompA and cryptic plasmid parallel
amplification method

| | | Internal Control |
|---|---|---|
| | C. trachomatis | (F3/Back-F1 or |
| Control Group | (F1 or 530) | 705/Black 530 |
| Escherichia coli | − | + |
| Candida glabrata | − | + |
| Candida albicans | − | + |
| Chlamydia psittaci | − | + |
| Chlamydia pneumoniae | − | + |
| Neisseria gonorrhoeae | − | + |
| Salmonella enteritides | − | + |
| Salmonella Typhimurium | − | + |
| Staphylococcus aureus | − | + |
| Streptococcus pyogenes | − | + |
| Streptococcus pneumoniae | − | + |
| Proteus mirabilis | − | + |
| Proteus vulgaris | − | + |
| Enterococus faecalis | − | + |
| Enterobacter coloacea | − | + |
| Klebsiella pneumoniae | − | + |
| Haemophilus partainfluenzae | − | + |
| Bordetella pertussis | − | + |
| Acinetobacter spp. | − | + |
| Gardnerella vaginalis | − | + |
| Herpes-simplex-Virus 1 und 2 | − | + |

Verification of robustness allows the determination of the total failure rate of the parallel amplification methods for detecting C. trachomatis. To determine robustness, parallel amplification assays were performed as described above, using primers specific for ompA and the cryptic plasmid. C. trachomatis negative samples of swabs, 30 of urine and 30 of semen were spiked with 2.1 copies/μl elution volume of C. trachomatis control DNA (approximately three-fold concentration of the analytical sensitivity limit). After extraction using the QIAamp DNA Mini Kit (seminal and swabs samples) or the MagNA Pure DNA Isolation Kit (urine, swabs, and seminal samples), these samples were analyzed by parallel amplification using probes specific for ompA and the cryptic plasmid. For all C. trachomatis samples, the failure rate was 0%. In addition, the robustness of the internal control was assessed by purification and analysis of 100 C. trachomatis negative swabs, 30 urine and 30 semen samples. The total failure rate was 0%. Inhibitions were not observed. Thus, the robustness of the parallel amplification methods using primers specific for ompA and the cryptic plasmid is ≧99%.

### Example 4

Precision of Parallel Amplification Detection of C. trachomatis Using Primers Specific for C. trachomatis ompA and Cryptic Plasmid

The total variance of the parallel amplification detection method was determined by collecting precision data using a LightCycler® 2.0 instrument via parallel amplification using primers specific for C. trachomatis genomic and cryptic plasmid sequences. Total variance consists of the intra-assay variability (variability of multiple results of samples of the same concentration within one experiment), the inter-assay variability (variability of multiple results of the assay generated on different instruments of the same type by different operators within one laboratory) and the inter-batch variability (variability of multiple results of the assay using various batches). The data obtained were used to determine the standard deviation, the variance, and the coefficient of variation for the pathogen specific and the internal control PCR.

Precision data were collected using the C. trachomatis serovar of the concentration that is next to the threefold cut-off value (2.1 copies/μl in consideration of the purification, 0.66 copies/μl without consideration of the purification). Testing was performed with eight replicates. The precision data was calculated on the basis of the Ct values of the amplification curves. Based on these results, the overall statistical spread of any given sample with the mentioned concentration was 3.95% (Ct), 3.89% (Ct) in consideration of the purification with the QIAamp DNA Mini Kit and 3.47% (Ct) in consideration of the MagNA Pure DNA Isolation Kit. For the detection of the internal control, the overall statistical spread was 2.29% (Ct), 3.48% (Ct) in consideration of the purification with the QIAamp DNA Mini Kit and 3.30% (Ct) in consideration of the MagNA Pure DNA Isolation Kit. These values were based on the totality of all single values of the determined variabilities. The results are shown in Tables 6-8.

TABLE 6
Precision Data on Basis of the Ct Values

| | | Standard | | Coefficient of |
|---|---|---|---|---|
| Assay | | Deviation | Variance | variation [%] |
| Intra-assay | variability: | 0.29 | 0.08 | 0.91 |
| C. trachomatis - | | | | |
| Serovar 0.66 | | | | |
| Intra-assay | variability: | 0.32 | 0.10 | 1.41 |
| Internal Control | | | | |
| Inter-assay | variability: | 0.74 | 0.55 | 2.41 |
| C. trachomatis - | | | | |
| Serovar 0.66 | | | | |
| Inter-assay | variability: | 0.36 | 0.13 | 1.58 |
| Internal Control | | | | |
| Inter-batch | variability: | 1.35 | 1.81 | 4.23 |
| C. trachomatis - | | | | |
| Serovar 0.66 | | | | |
| Inter-batch | variability: | 0.47 | 0.22 | 2.00 |
| Internal Control | | | | |
| Total | variance: | 1.16 | 1.35 | 3.95 |
| C. trachomatis - | | | | |
| Serovar 0.66 | | | | |
| Total | variance: | 0.59 | 0.34 | 2.29 |
| Internal Control | | | | |

TABLE 7
Precision Data in Consideration of QIAamp DNA Mini Kit

| | | | Standard | Coefficient of |
|---|---|---|---|---|
| Assay | | Deviation | Variance | variation [%] |
| Intra-assay | variability: | 0.14 | 0.02 | 0.46 |
| C. trachomatis - | | | | |
| Serovar 0.66 | | | | |
| Intra-assay | variability: | 0.29 | 0.08 | 1.18 |
| Internal Control | | | | |
| Inter-assay | variability: | 0.78 | 0.61 | 2.46 |
| C. trachomatis - | | | | |
| Serovar 0.66 | | | | |
| Inter-assay | variability: | 0.74 | 0.55 | 3.15 |
| Internal Control | | | | |
| Inter-batch | variability: | 1.11 | 1.23 | 3.33 |
| C. trachomatis - | | | | |
| Serovar 0.66 | | | | |
| Inter-batch | variability: | 0.64 | 0.41 | 2.62 |
| Internal Control | | | | |
| Total | variance: | 1.26 | 1.59 | 3.89 |
| C. trachomatis - | | | | |
| Serovar 0.66 | | | | |
| Total | variance: | 0.76 | 0.58 | 3.48 |
| Internal Control | | | | |

TABLE 8
Precision Data in Consideration of MagNA Pure DNA Isolation Kit

| | | Standard | | Coefficient of |
|---|---|---|---|---|
| Assay | | Deviation | Variance | variation [%] |
| Intra-assay | variability: | 0.14 | 0.02 | 0.44 |
| C. trachomatis - | | | | |
| Serovar 0.66 | | | | |
| Intra-assay | variability: | 0.28 | 0.08 | 1.10 |
| Internal Control | | | | |
| Inter-assay | variability: | 0.50 | 0.25 | 1.57 |
| C. trachomatis - | | | | |
| Serovar 0.66 | | | | |
| Inter-assay | variability: | 0.50 | 0.25 | 1.96 |
| Internal Control | | | | |
| Inter-batch | variability: | 1.39 | 1.93 | 4.17 |
| C. trachomatis - | | | | |
| Serovar 0.66 | | | | |
| Inter-batch | variability: | 0.83 | 0.69 | 3.30 |
| Internal Control | | | | |
| Total | variance: | 1.13 | 1.29 | 3.47 |
| C. trachomatis - | | | | |
| Serovar 0.66 | | | | |
| Total | variance: | 0.66 | 0.43 | 3.30 |
| Internal Control | | | | |

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

TABLE 9
Exemplary C. trachomatis polypeptide and polynucleotide sequences

| | *Nucleotide Accession | Brief | |
|---|---|---|---|
| *Protein Accession Number | Number | Description | Reference |
| gi\|144531\|gb\|AAA23141.1\| | gi\|144530\|gb\|M58938.1\| | strain | Hayes, L. J. and |
| | | A/SA1/OT; 396 | Clarke, I. N. 1990. |
| | | aa; C. trachomatis. | MEDLINE |
| | | See 129133. | 91045088; |
| | | | Hayes, L. J., |
| | | | Conlan, J. W., |
| | | | Everson, J. S. |
| | | | Ward, M. E. and |
| | | | Clarke, I. N. 1991. |
| | | | MEDLINE |
| | | | 92065225. |
| gi\|129133\|sp\|P23732\| | **none | serovar A; | Hayes, L. J. and |
| | | strain SA1/OT; | Clarke, I. N. 1990. |
| | | 396 aa; xrefs: | MEDLINE |
| | | gi: 144530, gi: | 91045088. |
| | | 144531, gi: | |
| | | 144546, gi: | |
| | | 144547, gi: | |
| | | 97280; C. trachomatis. | |
| gi\|144547\|gb\|AAA92785.1\| | gi\|144546\|gb\|M33635.1\| | serovar A; 396 | Hayes, L. J., |
| | | aa; C. trachomatis. | Pickett, M. A., |
| | | See 129133. | Conlan, J. W., |
| | | | Ferris, S., |
| | | | Everson, J. S., |
| | | | Ward, M. E. and |
| | | | Clarke, I. N. 1990. |
| | | | MEDLINE |
| | | | 91086917. |
| gi\|97280\|pir\|\|S12799\| | **none | serotype A; 396 | Hayes, L. J., |
| | | aa; C. trachomatis. | Pickett, M. A., |
| | | See 129133. | Conlan, J. W., |
| | | | Ferris, S., |
| | | | Everson, J. S. |
| | | | Ward, M. E. and |
| | | | Clarke, I. N. 1990. |
| | | | MEDLINE |
| | | | 91086917; |
| | | | Hayes, L. J. and |
| | | | Clarke, I. N. 1990. |
| | | | MEDLINE |
| | | | 91045088. |
| gi\|79374\|pir\|S11006\| | **none | serotype A; 374 | Baehr, W., |
| | | aa; C. trachomatis. | Zhang, Y. X., |
| | | | Joseph, T., Su, H., |
| | | | Nano, F. E., |
| | | | Everett, K. D. and |
| | | | Caldwell, H. D., 1988. |
| | | | MEDLINE |
| | | | 88234546. |
| gi\|144539\|gb\|AAA23145.1\| | gi\|144538\|gb\|J03813.1\| | serovar A; 396 | Baehr, W., |
| | | aa; C. trachomatis. | Zhang, Y. X., |
| | | | Joseph, T., Su, H., |
| | | | Nano, F. E., |
| | | | Everett, K. D. and |
| | | | Caldwell, H. D., 1988. |
| | | | MEDLINE |
| | | | 88234546. |
| gi\|129145\|sp\|P08780\| | **none | serovar C; 397 | Stephens, R. S., |
| | | aa, xrefs: gi: | Sanchez- |
| | | 144600, gi: | Pescador, R., |
| | | 144601, gi: | Wagar, E. A., |
| | | 72579; | Inouye, C. and |
| | | organism: C. trachomatis. | Urdea, M. S. 1987. |
| | | | MEDLINE |
| | | | 87307955. |
| gi\|72579\|pir\|\|MMCWTC\| | **none | serovar C; 397 | Stephens, R. S., |
| | | aa; C. trachomatis. | Sanchez- |
| | | See 129145 | Pescador, R., |
| | | | Wagar, E. A., |
| | | | Inouye, C. and |
| | | | Urdea, M. S. 1987. |
| | | | MEDLINE |
| | | | 87307955. |
| gi\|79376\|pir\|\|S11007\| | **none | serotype C; 375 | Baehr, W., |
| | | aa; C. trachomatis. | Zhang, Y. X., |
| | | | Joseph, T., Su, H., |
| | | | Nano, F. E., |
| | | | Everett, K. D. and |
| | | | Caldwell, H. D., 1988. |
| | | | MEDLINE |
| | | | 88234546. |
| gi\|144601\|gb\|AAA23156.1\| | gi\|144600\|gb\|M17343.1\| | serovar C; 397 | Stephens, R. S., |
| | | aa; C. trachomatis. | Wagar, E. A. and |
| | | See 129145 | Edman, U. 1988. |
| | | | MEDLINE |
| | | | 88115175. |
| gi\|3135639\|gb\|AAC31442.1\| | gi\|3135638\|gb\|AF063201.1\| | strain Ia/IU- | Stothard, D. R., |
| | | 4168; omp1; | Boguslawski, G. and |
| | | 397 aa; C. trachomatis. | Jones, R. B. 1998. |
| | | | MEDLINE |
| | | | 98339860. |
| gi\|3135647\|gb\|AAC31446.1\| | gi\|3135646\|gb\|AF063205.1\| | strain Ia/IU- | Stothard, D. R., |
| | | 1588; omp1; | Boguslawski, G. and |
| | | 317 aa; C. trachomatis. | Jones, R. B. 1998. |
| | | | MEDLINE |
| | | | 98339860. |
| gi\|3769545\|gb\|AAC64561.1\| | gi\|3769544\|gb\|AF086856.1\| | serotype J; | Stothard, D. R., Van |
| | | strain IU- | Der Pol, B., |
| | | 117542; | Smith, N. J. and |
| | | genotype J2; | Jones, R. B. “Effect |
| | | omp1; 388 aa; | of serial passage in |
| | | organism: C. trachomatis. | tissue culture on the |
| | | | sequence of omp1 |
| | | | from Chlamydia |
| | | | trachomatisclinical |
| | | | isolates”. |
| | | | Unpublished. |
| gi\|3135649\|gb\|AAC31447.1\| | gi\|3135648\|gb\|AF063206.1\| | strain J/IU- | Stothard, D. R., |
| | | 1553; omp1; C. trachomatis. | Boguslawski, G. and |
| | | | Jones, R. B. 1998. |
| | | | MEDLINE |
| | | | 98339860. |
| gi\|3135641\|gb\|AAC31443.1\| | gi\|3135640\|gb\|AF063202.1\| | strain | Stothard, D. R., |
| | | J/UW36/Cx; | Boguslawski, G. and |
| | | omp1; 397 aa; | Jones, R. B. 1998. |
| | | C. trachomatis. | MEDLINE |
| | | | 98339860. |
| gi\|3135637\|gb\|AAC31441.1\| | gi\|3135636\|gb\|AF063200.1\| | strain | Stothard, D. R., |
| | | I/UW12/Ur; | Boguslawski, G. and |
| | | omp1; C. trachomatis. | Jones, R. B. 1998. |
| | | | MEDLINE |
| | | | 98339860. |
| gi\|3135645\|gbAAC31445.1\| | gi\|3135644\|gb\|AF063204.1\| | strain | Stothard, D. R., |
| | | K/UW31/Cx; | Boguslawski, G. and |
| | | omp1; C. trachomatis. | Jones, R. B. 1998. |
| | | | MEDLINE |
| | | | 98339860. |
| gi\|3135651\|gb\|AAC31448.1\| | gi\|3135650\|gb\|AF063207.1\| | strain K/IU- | Stothard, D. R., |
| | | 1619; omp1; C. trachomatis. | Boguslawski, G. and |
| | | | Jones, R. B. 1998. |
| | | | MEDLINE |
| | | | 98339860. |
| gi\|4377420\|emb\|CAA34145.1\| | gi\|40720\|emb\|X16007.1\| | serovar H; 397 | Hamilton, P. T. and |
| | | aa; gi: 4377420 | Malinowski, D. P. |
| | | replaced gi: | 1989. MEDLINE |
| | | 40721; | 90045958. |
| | | organism: C. trachomatis. | |
| | | See 129152 | |
| gi\|129152\|sp\|P13467\| | **none | serovar H; 397 | Hamilton, P. T. and |
| | | aa; xrefs: gi: | Malinowski, D. P. |
| | | 40720, gi: | 1989. MEDLINE |
| | | 4377420, gi: | 90045958. |
| | | 72578; | |
| | | organism: C. trachomatis. | |
| gi\|72578\|pir\|\|MMCWTH\| | **none | serovar H; 397 | Hamilton, P. T. and |
| | | aa; C. trachomatis. | Malinowski, D. P. |
| | | See 129152. | 1989. MEDLINE |
| | | | 90045958. |
| gi\|3135643\|gb\|AAC31444.1\| | gi\|3135642\|gb\|AF063203.1\| | strain Ja/IU- | Stothard, D. R., |
| | | A795; identical | Boguslawski, G. and |
| | | to other Ja | Jones, R. B. 1998. |
| | | strains; 397 aa; | MEDLINE |
| | | omp1; | 98339860. |
| | | organism: C. trachomatis. | |
| gi\|2982485\|emb\|CAA06185.1\| | gi\|2982484\|emb\|AJ004876.1\| | isolate pm39; | Hoelzle, L. E., |
| | | 360 aa; omp1; | Steinhausen, G., |
| | | C. trachomatis. | Eggemann, G., |
| | | | Schiller, I. and |
| | | | Wittenbrink, M. M. |
| | | | “Isolation and |
| | | | Differentiation of |
| | | | Chlamydia spp. from |
| | | | swine”. |
| | | | Unpublished. |
| gi\|129156\|sp\|P23114\| | **none | serovar L3; | Fielder, T. J., |
| | | strain 404; 397 | Peterson, E. M. and |
| | | aa; xrefs: gi: | de la Maza, L. M. |
| | | 40709, gi: | 1991. MEDLINE |
| | | 40710, gi: | 91285429. |
| | | 79378; | |
| | | organism: C. trachomatis. | |
| gi\|40710\|emb\|CAA39226.1\| | gi\|40709\|emb\|X55700.1\| | serovar L3; | Fielder, T. J., |
| | | strain 404; 397 | Peterson, E. M. and |
| | | aa; organism: | de la Maza, L. M. |
| | | C. trachomatis. | 1991. MEDLINE |
| | | See 129156 | 91285429. |
| gi\|79378\|pir\|\|JE0413\| | **none | serotype L3; | Carlson, E. J., |
| | | 397 aa; C. trachomatis. | Peterson, E. M. and |
| | | See 129156 | de la Maza, L. M. |
| | | | 1989. MEDLINE |
| | | | 89108590; |
| | | | Kaul, R., |
| | | | Duncan, M. J., |
| | | | Guest, J. and |
| | | | Wenman, W. M. |
| | | | 1990. MEDLINE |
| | | | 90236304; |
| | | | Fielder, T. J., |
| | | | Peterson, E. M. and |
| | | | de la Maza, L. M. |
| | | | 1991. MEDLINE |
| | | | 91285429. |
| gi\|3135659\|gb\|AAC31452.1\| | gi\|3135658\|gb\|AF063211.1\| | strain E/IU- | Stothard, D. R., |
| | | 1579; identical | Boguslawski, G. and |
| | | to E/Bour | Jones, R. B. 1998. |
| | | reference | MEDLINE |
| | | strain; >313 aa; | 98339860. |
| | | omp1; | |
| | | organism: C. trachomatis. | |
| gi\|1938316\|gb\|AAC45149.1\| | gi\|1938315\|gb\|U78534.1\| | serotype E; | Dean, D. and |
| | | Allele E/33; 381 | Millman, K. 1997. |
| | | aa; omp1; | MEDLINE |
| | | organism: C. trachomatis. | 97174347. |
| gi\|1938326\|gb\|AAC45154.1\| | gi\|1938325\|gb\|U78763.1\| | serovar E; | Dean, D. and |
| | | strain E/Bour; | Millman, K. 1997. |
| | | 381 aa; omp1; | MEDLINE |
| | | organism: C. trachomatis. | 97174347. |
| gi\|1938310\|gb\|AAC45146.1\| | gi\|1938309\|gb\|U78531.1\| | serotype E; | Dean, D. and |
| | | allele E/15; 381 | Millman, K. 1997. |
| | | aa; omp1; C. trachomatis. | MEDLINE |
| | | | 97174347. |
| gi\|1938314\|gb\|AAC45148.1\| | gi\|1938313\|gb\|U78533.1\| | serotype E; | Dean, D. and |
| | | allele E/25; 381 | Millman, K. 1997. |
| | | aa; omp1; C. trachomatis. | MEDLINE |
| | | | 97174347. |
| gi\|1938308\|gb\|AAC45145.1\| | gi\|1938307\|gb\|U78530.1\| | serotype E; | Dean, D. and |
| | | allele E/3; 381 | Millman, K. 1997. |
| | | aa; omp1; C. trachomatis. | MEDLINE |
| | | | 97174347. |
| gi\|1938306\|gb\|AAC45144.1\| | gi\|1938305\|gb\|U78529.1\| | serotype E; | Dean, D. and |
| | | allele E/2; 381 | Millman, K. 1997. |
| | | aa; omp1; C. trachomatis. | MEDLINE |
| | | | 97174347. |
| gi\|1938304\|gb\|AAC45143.1\| | gi\|1938303\|gb\|U78528.1\| | serotype E; | Dean, D. and |
| | | allele E/1; 381 | Millman, K. 1997. |
| | | aa; omp1; C. trachomatis. | MEDLINE |
| | | | 97174347. |
| gi\|1938312\|gb\|AAC45147.1\| | gi\|1938311\|gb\|U78532.1\| | serotype E; | Dean, D. and |
| | | allele E/18; 381 | Millman, K. 1997. |
| | | aa; omp1; C. trachomatis. | MEDLINE |
| | | | 97174347. |
| gi\|1938320\|gb\|AAC45151.1\| | gi\|1938319\|gb\|U78536.1\| | serotype E; | Dean, D. and |
| | | allele E/48; 381 | Millman, K. 1997. |
| | | aa; omp1; C. trachomatis. | MEDLINE |
| | | | 97174347. |
| gi\|1938324\|gb\|AAC45153.1\| | gi\|1938323\|gb\|U78538.1\| | serotype E; | Dean, D. and |
| | | allele E/67; 381 | Millman, K. 1997. |
| | | aa; omp1; C. trachomatis. | MEDLINE |
| | | | 97174347. |
| gi\|1938318\|gb\|AAC45150.1\| | gi\|1938317\|gb\|U78535.1\| | serotype E; | Dean, D. and |
| | | allele E/43; 381 | Millman, K. 1997. |
| | | aa; omp1; C. trachomatis. | MEDLINE |
| | | | 97174347. |
| gi\|1938322\|gb\|AAC45152.1\| | gi\|1938321\|gb\|U78537.1\| | serotype E; | Dean, D. and |
| | | allele E/55; 381 | Millman, K. 1997. |
| | | aa; omp1; C. trachomatis. | MEDLINE |
| | | | 97174347. |
| gi\|3135657\|gb\|AAC31451.1\| | gi\|3135656\|gb\|AF063210.1\| | strain E/IU- | Stothard, D. R., |
| | | 1586; identical | Boguslawski, G. and |
| | | to E/Bour | Jones, R. B. 1998. |
| | | reference | MEDLINE |
| | | strain; >313 aa; | 98339860. |
| | | omp1; C. trachomatis. | |
| gi\|129149\|sp\|P17451\| | **none | serovar E; 393 | Peterson, E. M., |
| | | aa; xrefs: gi: | Markoff, B. A. and de |
| | | 40713, gi: | la Maza, L. M. 1990. |
| | | 40714, gi: | MEDLINE |
| | | 72580; C. trachomatis. | 90287737. |
| gi\|40714\|emb\|CAA36791.1\| | gi\|40713\|emb\|X52557.1\| | serovar E; | Peterson, E. M., |
| | | strain Bour; 393 | Markoff, B. A. and de |
| | | aa; organism: | la Maza, L. M. 1990. |
| | | C. trachomatis. | MEDLINE |
| | | See 129149 | 90287737. |
| gi\|72580\|pir\|\|MMCWTE\| | **none | serotype E; 393 | Peterson, E. M., |
| | | aa; C. trachomatis. | Markoff, B. A. and de |
| | | See 129149 | la Maza, L. M. 1990. |
| | | | MEDLINE |
| | | | 90287737. |
| gi\|3135633\|gb\|AAC31439.1\| | gi\|3135632\|gb\|AF063198.1\| | strain E/IU- | Stothard, D. R., |
| | | 51538; identical | Boguslawski, G. and |
| | | to strains: E/IU- | Jones, R. B. 1998. |
| | | 71253; −71254; | MEDLINE |
| | | 393 aa; omp1; | 98339860. |
| | | C. trachomatis. | |
| gi\|144599\|gb\|AAA23155.1\| | gi\|144598\|gb\|M17342.1\| | serotype B; | Stephens, R. S., |
| | | strain B/TW- | Wagar, E. A. and |
| | | 5/OT; 394 aa; | Edman, U. 1988. |
| | | C. trachomatis. | MEDLINE |
| | | See 129144 | 88115175. |
| gi\|79375\|pir\|\|S11009\| | **none | serotype B; 372 | Baehr, W., |
| | | aa; C. trachomatis. | Zhang, Y. X., |
| | | | Joseph, T., Su, H., |
| | | | Nano, F. E., |
| | | | Everett, K. D. and |
| | | | Caldwell, H. D., 1988. |
| | | | MEDLINE |
| | | | 88234546. |
| gi\|129144\|sp\|P23421\| | **none | serotype B; 394 | Stephens, R. S., |
| | | aa; xrefs: gi: | Sanchez- |
| | | 144598, gi: | Pescador, R., |
| | | 72581; C. trachomatis. | Wagar, E. A., |
| | | | Inouye, C. and |
| | | | Urdea, M. S. 1987. |
| | | | MEDLINE |
| | | | 87307955. |
| gi\|72581\|pir\|\|MMCWTB\| | **none | serotype B; 394 | Stephens, R. S., |
| | | aa; C. trachomatis. | Sanchez- |
| | | See 129144 | Pescador, R., |
| | | | Wagar, E. A., |
| | | | Inouye, C. and |
| | | | Urdea, M. S. 1987. |
| | | | MEDLINE |
| | | | 87307955. |
| gi\|320451\|pir\|\|B60756\| | **none | serotype B; 372 | Hayes, L. J., |
| | | aa; C. trachomatis. | Pickett, M. A., |
| | | | Conlan, J. W., |
| | | | Ferris, S., |
| | | | Everson, J. S., |
| | | | Ward, M. E. and |
| | | | Clarke, I. N. 1990. |
| | | | MEDLINE |
| | | | 91086917. |
| gi\|3135653\|gb\|AAC31449.1\| | gi\|3135652\|gb\|AF063208.1\| | strain B/IU- | Stothard, D. R., |
| | | 1226; identical | Boguslawski, G. and |
| | | to Bb strain; | Jones, R. B. 1998. |
| | | omp1; >314 aa; | MEDLINE |
| | | C. trachomatis. | 98339860. |
| gi\|144549\|gb\|AAA92784.1\| | gi\|144548\|gb\|M33636.1\| | serovar B; 394 | Hayes, L. J., |
| | | aa; C. trachomatis. | Pickett, M. A., |
| | | | Conlan, J. W., |
| | | | Ferris, S., |
| | | | Everson, J. S., |
| | | | Ward, M. E. and |
| | | | Clarke, I. N. 1990. |
| | | | MEDLINE |
| | | | 91086917. |
| gi\|3135625\|gb\|AAC31435.1\| | gi\|3135624\|gb\|AF063194.1\| | strain | Stothard, D. R., |
| | | Ba/Apache-2; | Boguslawski, G. and |
| | | 394 aa; C. trachomatis. | Jones, R. B. 1998. |
| | | | MEDLINE |
| | | | 98339860. |
| gi\|2076876\|gb\|AAB54112.1\| | gi\|2076875\|gb\|U80075.1\| | serotype B; | Farencena, A., |
| | | strain alfa/95; | Comanducci, M., |
| | | 394 aa; omp1; | Donati, M., |
| | | immunogenic | Cevenini, R. and |
| | | surface protein | Ratti, G. 1997. |
| | | with 4 VD and 5 | MEDLINE |
| | | CD; C. trachomatis. | 97342776. |
| gi\|2956655\|emb\|CAA54553\| | gi\|2956654\|emb\|X77364.1\| | serovar D; | Sayada, C., |
| | | strain D/Ep6; | Vretou, E., Orfila, J., |
| | | 336 aa; omp1; | Elion, J. and |
| | | C. trachomatis. | Denamur, E. 1995. |
| | | | MEDLINE |
| | | | 96098951. |
| gi\|6707730\|gi\|6707730\| | **none | strain D/UW- | Stephens, R. S., |
| | | 3/CX; 393 aa; | Kalman, S., |
| | | ompA (see | Lammel, C. J., |
| | | CT681); C. trachomatis. | Fan, J., Marathe, R., |
| | | xrefs: gi: | Aravind, L., |
| | | 1143430, gi: | Mitchell, W. P., |
| | | 1143431, gi: | Olinger, L., |
| | | 3135626, gi: | Tatusov, R. L., |
| | | 3135627, gi: | Zhao, Q., |
| | | 3329126, gi: | Koonin, E. V. and |
| | | 3329133. | Davis, R. W. 1998. |
| | | | MEDLINE |
| | | | 99000809; |
| | | | Stothard, D. R., |
| | | | Boguslawski, G. and |
| | | | Jones, R. B. 1998. |
| | | | MEDLINE |
| | | | 98339860; |
| | | | Sayada, C., |
| | | | Denamur, E. and |
| | | | Elion, J. 1992. |
| | | | MEDLINE |
| | | | 93013014. |
| gi\|3329133\|gb\|AAC68276.1\| | gi\|3329126\|gb\|AE001338.1\| | strain D/UW- | Stephens, R. S., |
| | | 3/CX; 393 aa; | Kalman, S., |
| | | ompA (see | Lammel, C. J., |
| | | CT681); C. trachomatis. | Fan, J., Marathe, R., |
| | | See 6707730. | Aravind, L., |
| | | | Mitchell, W. P., |
| | | | Olinger, L., |
| | | | Tatusov, R. L., |
| | | | Zhao, Q., |
| | | | Koonin, E. V. and |
| | | | Davis, R. W. 1998. |
| | | | MEDLINE |
| | | | 99000809. |
| gi\|3135627\|gb\|AAC31436.1\| | gi\|3135626\|gb\|AF063195.1\| | strain D/IU- | Stothard, D. R. |
| | | 71960; identical | Boguslawski, G. and |
| | | to strains D/IU- | Jones, R. B. 1998. |
| | | 88712; −38638; | MEDLINE |
| | | −87178; 393 aa; | 98339860. |
| | | omp1; C. trachomatis. | |
| | | See 6707730. | |
| gi\|3769543\|gb\|AAC64560.1\| | gi\|3769542\|gb\|AF086855.1\| | serotype D; | Stothard, D. R., Van |
| | | strain IU- | Der Pol, B., |
| | | 116293; | Smith, N. J. and |
| | | genotype D6; | Jones, R. B. “Effect |
| | | >384 aa; C. trachomatis. | of serial passage in |
| | | | tissue culture on the |
| | | | sequence of omp1 |
| | | | from Chlamydia |
| | | | trachomatis clinical |
| | | | isolates”. |
| | | | Unpublished. |
| gi\|1321799\|emb\|CAA54554\| | gi\|1321798\|emb\|X77365.1\| | serovar Da; | Sayada, C., |
| | | strain Da/Ev- | Vretou, E., Orfila, J., |
| | | 293; omp1; C. trachomatis. | Elion, J. and |
| | | | Denamur, E. 1995. |
| | | | MEDLINE |
| | | | 96098951. |
| gi\|3135629\|gb\|AAC31437.1\| | gi\|3135628\|gb\|AF063196.1\| | strain D/IU- | Stothard, D. R., |
| | | 72403; 393 aa; | Boguslawski, G. and |
| | | C. trachomatis. | Jones, R. B. 1998. |
| | | | MEDLINE |
| | | | 98339860. |
| gi\|3769541\|gb\|AAC64559.1\| | gi\|3769540\|gb\|AF086854.1\| | serotype D; | Stothard, D. R., Van |
| | | strain IU- | Der Pol, B., |
| | | 116015; >384 | Smith, N. J. and |
| | | aa; omp1; C. trachomatis. | Jones, R. B. “Effect |
| | | | of serial passage in |
| | | | tissue culture on the |
| | | | sequence of omp1 |
| | | | from Chlamydia |
| | | | trachomatis clinical |
| | | | isolates”. |
| | | | Unpublished. |
| gi\|3135655\|gb\|AAC31450.1\| | gi\|3135654\|gb\|AF063209.1\| | strain Da/IU- | Stothard, D. R., |
| | | 1554; >313 aa; | Boguslawski, G. and |
| | | omp1; C. trachomatis. | Jones, R. B. 1998. |
| | | | MEDLINE |
| | | | 98339860. |
| gi\|3135631\|gb\|AAC31438.1\| | gi\|3135630\|gb\|AF063197.1\| | strain D/IU- | Stothard, D. R., |
| | | 83786; identical | Boguslawski, G. and |
| | | to other D | Jones, R. B. 1998. |
| | | strains; 393 aa; | MEDLINE |
| | | omp1; C. trachomatis. | 98339860. |
| gi\|143435\|emb\|CAA44703.1\| | gi\|143434\|emb\|X62920.1\| | serovar D; | Sayada, C., |
| | | strain D/IC-Cal- | Denamur, E. and |
| | | 8; 393 aa; | Elion, J. 1992. |
| | | omp1; C. trachomatis. | MEDLINE |
| | | | 93013014. |
| gi\|143431\|emb\|CAA44701.1\| | gi\|1143430\|emb\|X62918.1\| | serovar D, | Sayada, C., |
| | | strain D/B-120; | Denamur, E. and |
| | | 393 aa; omp1; | Elion, J. 1992. |
| | | C. trachomatis. | MEDLINE |
| | | See 6707730. | 93013014. |
| gi\|11434331\|emb\|CAA44702\| | gi\|1143432\|emb\|X62919.1\| | serovar D; | Sayada, C., |
| | | strain D/B-185; | Denamur, E. and |
| | | 393 aa; omp1; | Elion, J. 1992. |
| | | C. trachomatis. | MEDLINE |
| | | | 93013014. |
| gi\|40719\|emb\|CAA4474.1\| | gi\|40718\|emb\|X62921.1\| | serovar D; | Sayada, C., |
| | | strain Da/TW- | Denamur, E. and |
| | | 448; 393 aa; | Elion, J. 1992. |
| | | omp1; C. trachomatis. | MEDLINE |
| | | | 93013014. |
| gi\|321976\|pir\|\|JC1432\| | **none | serotype Da; | Sayada, C., |
| | | 393 aa; C. trachomatis. | Denamur, E. and |
| | | | Elion, J. 1992. |
| | | | MEDLINE |
| | | | 93013014. |
| gi\|144533\|gb\|AAA23142.1\| | gi\|144532\|gb\|M36533.1\| | serotype L1; | Pickett, M. A., |
| | | 393 aa; C. trachomatis. | Ward, M. E. and |
| | | See 129154. | Clarke, I. N. 1987. |
| gi\|129154\|sp\|P19542\| | **none | serovar L1; 393 | Pickett, M. A., |
| | | aa; xrefs: gi: | Ward, M. E. and |
| | | 144532, gi: | Clarke I. N. 1987. |
| | | 144533, gi: | |
| | | 79379; C. trachomatis. | |
| gi\|79379\|gb\|AAA23142.1\| | **none | serotype L1; | Pickett, M. A., |
| | | 393 aa; C. trachomatis. | Ward, M. E. and |
| | | See 129154. | Clarke, I. N. 1987. |
| gi\|129155\|sp\|P06597\| | **none | serovar L2; 394 | Stephens, R. S., |
| | | aa; xrefs: gi: | Mullenbach, G., |
| | | 144550, gi: | Sanchez- |
| | | 144551, gi: | Pescador, R. and |
| | | 97273; C. trachomatis. | Agabian, N. 1986. |
| | | | MEDLINE |
| | | | 87057033. |
| gi\|144551\|gb\|AAA23151.1\| | gi\|144550\|gb\|M14738.1\| | serovar L2; | Stephens, R. S., |
| | | omp1; 394 aa; | Wagar, E. A. and |
| | | C. trachomatis. | Edman, U. 1988. |
| | | See 129155. | MEDLINE |
| | | | 88115175. |
| gi\|97273\|pir\|\|S11012\| | **none | C. trachomatis. | Nano, F. E., |
| | | See 129155. | Barstad, P. A., |
| | | | Mayer, L. W., |
| | | | Coligan, J. E. and |
| | | | Caldwell, H. D. 1985. |
| | | | MEDLINE |
| | | | 85181625; |
| | | | Stephens, R. S., |
| | | | Mullenbach, G., |
| | | | Sanchez- |
| | | | Pescador, R. and |
| | | | Agabian, N. 1986. |
| | | | MEDLINE |
| | | | 87057033; |
| | | | Baehr, W., |
| | | | Zhang, Y. X., |
| | | | Joseph, T., Su, H., |
| | | | Nano, F. E., |
| | | | Everett, K. D. and |
| | | | Caldwell, H. D. 1988. |
| | | | MEDLINE |
| | | | 88234546. |
| gi\|532120\|gb\|AAA23149.1\| | gi\|532119\|gb\|L35605.1\| | strain LGV; | Hayes, L. J. 1994. |
| | | isolate 98; 393 | |
| | | aa; omp1; C. trachomatis. | |
| gi\|532122\|gb\|AAA23150.1\| | gi\|532121\|gb\|L35606.1\| | strain LGV; | Hayes, L. J. 1994. |
| | | isolate 115; 393 | |
| | | aa; omp1; C. trachomatis. | |
| gi\|3135663\|gb\|AAC31454.1\| | gi\|3135662\|gb\|AF063213.1\| | strain F/IU- | Stothard, D. R., |
| | | 1607; 315 aa; | Boguslawski, G. and |
| | | note: STD urine | Jones, R. B. 1998. |
| | | isolate; identical | MEDLINE |
| | | to F reference | 98339860. |
| | | strain; C. trachomatis. | |
| gi\|3135661\|gb\|AAC31453.1\| | gi\|3135660\|gb\|AF063212.1\| | strain F/IU- | Stothard, D. R., |
| | | 1552; >315 aa; | Boguslawski, G. and |
| | | C. trachomatis. | Jones, R. B. 1998. |
| | | | MEDLINE |
| | | | 98339860. |
| gi\|129150\|sp\|P16155\| | **none | serovar F; 395 | Zhang, Y. X., |
| | | aa; xrefs: gi: | Morrison, S. G., |
| | | 40705, gi: | Caldwell, H. D. 1990. |
| | | 40706, gi: | MEDLINE |
| | | 72582; C. trachomatis. | 90192102. |
| gi\|40706\|emb\|CAA36299.1\| | gi\|40705\|emb\|X52080.1\| | serovar F; | Zhang, Y. X., |
| | | strain IC-Cal3; | Morrison, S. G., |
| | | 395 aa; C. trachomatis. | Caldwell, H. D. 1990. |
| | | See 129150. | MEDLINE |
| | | | 90192102. |
| gi\|72582\|pir\|\|MMCWTF\| | **none | serovar F; 395 | Zhang, Y. X., |
| | | aa; C. trachomatis. | Morrison, S. G., |
| | | See 129150. | Caldwell, H. D. 1990. |
| | | | MEDLINE |
| | | | 90192102. |
| gi\|3135635\|gb\|AAC31440.1\| | gi\|3135634\|gb\|AF063199.1\| | strain | Stothard, D. R., |
| | | G/UW57/Cx; | Boguslawski, G. and |
| | | 395 aa; omp1; | Jones, R. B. 1998. |
| | | C. trachomatis. | MEDLINE |
| | | | 98339860. |
| gi\|79380\|pir\|\|JT0947\| | **none | mouse | Fielder, T. J., Pal, S., |
| | | pneumonitis | Peterson, E. M. and |
| | | biovar MoPn; | de la Maza, L. M. |
| | | 387 aa; | 1991. MEDLINE |
| | | Chlamydie | 92039057. |
| | | trachomatis. | |
| gi\|97281\|pir\|\|S16034\| | **none | 387 aa; C. trachomatis. | Peterson, E. M., |
| | | | Cheng, X., |
| | | | Markoff, B. A., |
| | | | Fielder, T. J. and de |
| | | | la Maza, L. M. 1991. |
| | | | MEDLINE |
| | | | 92040090. |
| gi\|2119833\|pir\|\|40741\| | **none | 404 aa; C. trachomatis. | Zhang, Y. X., |
| | | | Fox, J. G., Ho, Y., |
| | | | Zhang, L., Stills, H. F. |
| | | | and Smith, T. F. |
| | | | 1993. MEDLINE |
| | | | 94104488. |
| gi\|40565\|emb\|CAA43409.1\| | gi\|40564\|emb\|X61096.1\| | strain | Storey, C., |
| | | FPn/pring; 392 | Lusher, M., Yates, P. |
| | | aa; organism: | and Richmond, S. |
| | | Chlamydophila | 1993. MEDLINE |
| | | felis; submitted | 94103736. |
| | | as Chlamydia | |
| | | psittaci. See | |
| | | 129130. | |
| gi\|129126\|sp\|P10332\| | **none | strain EAE | Pickett, M. A., |
| | | A22/M; 402 aa; | Everson, S. J. and |
| | | xrefs: gi: 40604, | Clarke, I. N. 1988. |
| | | gi: 40605, gi: | |
| | | 144542, gi: | |
| | | 144543, gi: | |
| | | 72584; | |
| | | organism: | |
| | | Chlamydia | |
| | | psittaci. | |
| gi\|40605\|emb\|CAA31177.1\| | gi\|40604\|emb\|X12647.1\| | strain EAE | Pickett, M. A., |
| | | A22/M; 402 aa; | Everson, S. J. and |
| | | organism: | Clarke, I. N. 1988. |
| | | Chlamydia | |
| | | psittaci. See | |
| | | 129126. | |
| gi\|72584\|pir\|\|MMCWPM\| | **none | 402 aa; | Pickett, M. A., |
| | | organism: | Everson, S. J. and |
| | | Chlamydia | Clarke, I. N. 1988. |
| | | psittaci. See | |
| | | 129126. | |
| gi\|6707728\|sp\|P75024\| | **none | mouse | Read, T. D., et al., |
| | | pneumonitis | 2000. MEDLINE |
| | | strain MoPn; | 10684935; |
| | | 387 aa; xrefs: | Fielder, T. J., Pal, S., |
| | | gi: 144536, gi: | Peterson, E. M. and |
| | | 144537, gi: | de la Maza, L. M. |
| | | 1518658, gi: | 1991. MEDLINE |
| | | 1518659, gi: | 92039057. |
| | | 927404, gi: | |
| | | 927405, gi: | |
| | | 144571, gi: | |
| | | 4757611; | |
| | | organism: | |
| | | Chlamydia | |
| | | muridarum. | |
| gi\|144537\|gb\|AAA23144.1\| | gi\|144536\|gb\|M64171.1\| | mouse | Fielder, T. J., Pal, S., |
| | | pneumonitis | Peterson, E. M. and |
| | | strain MoPn; | de la Maza, L. M. |
| | | 387 aa; | 1991. MEDLINE |
| | | Chlamydia | 92039057. |
| | | muridarum. See | |
| | | 6707728. | |
| gi\|1518659\|gb\|AAB07068.1\| | gi\|1518658\|gb\|U60196.1\| | mouse | Zhang, Y. -X., Tao, J., |
| | | pneumonitis | Zhou, M., Meng, Q., |
| | | strain MoPn; | Zhang, L. and |
| | | 387 aa; C. muridarum; | Miller, D. L. “Cloning, |
| | | submitted as | Sequencing, and |
| | | Chlamydia | Expression in E. coli |
| | | trachomatis. | of the Gene |
| | | See 6707728. | Encoding |
| | | | Elongation Factor |
| | | | Ts of C. trachomatis”. |
| | | | Unpublished. |
| gi\|410147\|gb\|AAA16615.1\| | gi\|410146\|gb\|L19221.1\| | strain SFPD; | Zhang, Y. X., |
| | | 404 aa; C. muridarum | Fox, J. G., Ho, Y., |
| | | | Zhang, L., Stills, H. F. |
| | | | and Smith, T. F. |
| | | | 1993. MEDLINE |
| | | | 94104488. |
| gi\|927405\|emb\|CAA45006.1\| | gi\|927404\|emb\|X63409.1\| | strain MoPn; | Carter, M. W., |
| | | 387aa; C. muridarum. | Giles, I., |
| | | See | Everson, J. S. and |
| | | 6707728. | Clarke, I. N. 1992. |
| gi\|4757611\|gb\|AAD29101.1\| | gi\|144571\|gb\|M73044.1\| | strain MoPn; | Kaltenboeck, B., |
| | | >339aa; | Kousoulas, K. G. and |
| | | organism: | Storz, J. 1993. |
| | | Chlamydophila | MEDLINE |
| | | muridarum. See | 93123168. |
| | | 6707728. | |
| gi\|5032264\|gb\|AAD38212.1\| | gi\|144569\|gb\|M73042.1\| | Chlamydophila | Kaltenboeck, B., |
| | | pecorum; >340 | Kousoulas, K. G. and |
| | | aa; isolate | Storz, J. 1993. |
| | | LW613; | MEDLINE |
| | | submitted as | 93123168. |
| | | Chlamydia | |
| | | psittaci. | |
| gi\|4928270\|gb\|AAD33511.1\| | gi\|4928269\|gb\|AF131230.1\| | mutant MOMP; | Tharp, A. C., |
| | | >223 aa; similar | Mitchell, W. M., |
| | | to GenBank | Stratton, C. W. and |
| | | Accessions | Ding, L. -M. |
| | | M64064, | “Presence of viable |
| | | M34942, and | Chlamydia |
| | | M64063; C. pneumoniae. | pneumoniae in fetal |
| | | | calf serum and in |
| | | | epithelial-derived |
| | | | cell lines”. |
| | | | Unpublished. |
| gi\|144535\|gb\|AAA23143.1\| | gi\|144534\|gb\|M64064.1\| | 389 aa; C. pneumoniae. | Carter, M. W., al- |
| | | See 129125. | Mahdawi, S. A., |
| | | | Giles, I. G., |
| | | | Treharne, J. D., |
| | | | Ward, M. E. and |
| | | | Clark, I. N. 1991. |
| | | | MEDLINE |
| | | | 91237311. |
| gi\|4928268\|gb\|AAD33510.1\| | gi\|4928267\|gb\|AF131229.1\| | mutant MOMP; | Tharp, A. C., |
| | | >223 aa; similar | Mitchell, W. M., |
| | | to GenBank | Stratton, C. W. and |
| | | Accessions | Ding, L. -M. |
| | | M64064, | “Presence of viable |
| | | M34942, and | Chlamydia |
| | | M64063; C. pneumoniae. | pneumoniae in fetal |
| | | | calf serum and in |
| | | | epithelial-derived |
| | | | cell lines”. |
| | | | Unpublished. |
| gi\|280182\|pir\|\|A43587\| | **none | 389 aa; ompA; | Carter, M. W., al- |
| | | C. pneumoniae. | Mahdawi, S. A., |
| | | See 129125. | Giles, I. G., |
| | | | Treharne, J. D., |
| | | | Ward, M. E. and |
| | | | Clark, I. N. 1991. |
| | | | MEDLINE |
| | | | 91237311; |
| | | | Perez Melgosa, M., |
| | | | Kuo, C. C. and |
| | | | Campbell, L. A. 1991. |
| | | | MEDLINE |
| | | | 91244474; |
| | | | Gaydos, C. A., |
| | | | Quinn, T. C., |
| | | | Bobo, L. D. and |
| | | | Eiden, J. J. 1992. |
| | | | MEDLINE |
| | | | 93084388. |
| gi\|6707729\|sp\|Q07430\| | **none | 389 aa; strain: | Storey, C., |
| | | N16; xrefs: gi: | Lusher, M., Yates, P. |
| | | 289840, gi: | and Richmond, S. |
| | | 289841; | 1993. MEDLINE |
| | | specific host: | 94103736. |
| | | Equus caballus; | |
| | | C. pneumoniae. | |
| gi\|289841\|gb\|AAA17397.1\| | gi\|289840\|gb\|L04982.1\| | 389 aa; specific | Storey, C., |
| | | host: Equus | Lusher, M., Yates, P. |
| | | caballus; C. pneumoniae. | and Richmond, S. |
| | | See 6707729. | 1993. MEDLINE |
| | | | 94103736. |
| gi\|129125\|sp\|P27455\| | **none | 389 aa; xrefs: | Carter, M. W., al- |
| | | gi: 144534, gi: | Mahdawi, S. A., |
| | | 144535, gi: | Giles, I. G., |
| | | 144540, gi: | Treharne, J. D., |
| | | 144541, gi: | Ward, M. E. and |
| | | 280182; C. pneumoniae. | Clark, I. N. 1991. |
| | | | MEDLINE |
| | | | 91237311; |
| | | | Perez Melgosa, M., |
| | | | Kuo, C. C. and |
| | | | Campbell, L. A. 1991. |
| | | | MEDLINE |
| | | | 91244474. |
| gi\|144541\|gb\|AAA73071.1\| | gi\|144540\|gb\|M69230.1\| | 389 aa; | Perez Melgosa, M., |
| | | Chlamydia | Kuo, C. -C. and |
| | | pneumoniae. | Campbell, L. A. 1991. |
| | | See 129125. | MEDLINE |
| | | | 91244474. |
| gi\|4545321\|gb\|AAD22492.1\| | gi\|4545320\|gb\|AF131889.1\| | 389 aa; C. pneumoniae. | Sriram, S., |
| | | | Mitchell, W. M. and |
| | | | Stratton, C. W. 1998. |
| | | | MEDLINE |
| | | | 98145402</a. |
| gi\|5032262\|gb\|AAD38210.1\| | gi\|144566\|gb\|M73038.1\| | >333 aa; | Kaltenboeck, B., |
| | | Chlamydophila | Kousoulas, K. G. and |
| | | pneumoniae; | Storz, J. 1993. |
| | | submitted as | MEDLINE |
| | | Chlamydia | 93123168. |
| | | psittaci. | |
| gi\|144543\|gb\|AAA23146.1\| | gi\|144542\|gb\|M36703.1\| | 402 aa; C. psittaci. | Pickett, M. A., |
| | | See | Everson, J. S. and |
| | | 129126. | Clarke, I. N. 1988. |
| gi\|144545\|gb\|AAA17396.1\| | gi\|144544\|gb\|L04980.1\| | 402 aa; C. psittaci. | Storey, C., |
| | | | Lusher, M., Yates, P. |
| | | | and Richmond, S. |
| | | | 1993. MEDLINE |
| | | | 94103736. |
| gi\|40569\|emb\|CAA40300.1\| | gi\|40568\|emb\|X56980.1\| | strain 6BC; 402 | Everett, K. D., |
| | | aa; C. psittaci. | Andersen, A. A., |
| | | | Plaunt, M. and |
| | | | Hatch, T. P. 1991. |
| | | | MEDLINE |
| | | | 91310346. |
| gi\|457206\|gb\|AAA23148.1\| | gi\|457205\|gb\|L25437.1\| | strain Avian | Storey, C. C. |
| | | type C; 402 aa; | “Analysis of the |
| | | C. psittaci. | MOMP gene of |
| | | | avian Chlamydia |
| | | | psittaci types A, C |
| | | | and E”. Unpublished |
| | | | (1994). |
| gi\|129128\|sp\|P16567\| | **none | 389 aa; xrefs: | Herring, A. J., |
| | | gi: 40600, gi: | Tan T. W., Baxter, S., |
| | | 40601, gi: | Inglis, N. F. and |
| | | 662830, gi: | Dunbar, S. 1989. |
| | | 625121, gi: | MEDLINE |
| | | 72583; C. psittaci. | 90128177; |
| | | | Griffiths, P. C., |
| | | | Plater, J. M., |
| | | | Martin, T. C., |
| | | | Hughes, S. L., |
| | | | Hughes, K. J., |
| | | | Hewinson, R. G. and |
| | | | Dawson, M. 1995. |
| | | | MEDLINE |
| | | | 96189695. |
| gi\|40601\|emb\|CAA36152.1\| | gi\|40600\|emb\|X51859.1\| | 389 aa; omp1; | Herring, A. J., |
| | | strain: S26/3; | Tan, T. W., Baxter, S., |
| | | specific host: | Inglis, N. F. and |
| | | Ovis aries; C. abortus; | Dunbar, S. 1989. |
| | | submitted as | MEDLINE |
| | | Chlamydia | 90128177. |
| | | psittaci. See | |
| | | 129128. | |
| gi\|625121\|gb\|AAB02850.1\| | gi\|662830\|gb\|L39020.1\| | 389 aa; ompA; | Griffiths, P. C., |
| | | strain: BA1; | Plater, J. M., |
| | | specific host: | Martin, T. C., |
| | | Bos taurus; C. abortus; | Hughes, S. L., |
| | | submitted as | Hughes, K. J., |
| | | Chlamydia | Hewinson, R. G. and |
| | | psittaci. See | Dawson, M. 1995. |
| | | 129128. | MEDLINE |
| | | | 96189695. |
| gi\|72583\|pir\|\|MMCWP3\| | **none | 389 aa; strain: | Herring, A. J., |
| | | S26/3; C. psittaci. | Tan, T. W., Baxter, S., |
| | | See | Inglis, N. F. and |
| | | 129128. | Dunbar, S. 1989. |
| | | | MEDLINE |
| | | | 90128177. |
| gi\|457204\|gb\|AAA23147.1\| | gi\|457203\|gb\|L25436.1\| | strain Avian | Storey, C. C. |
| | | type C; 391 aa; | “Analysis of the |
| | | C. psittaci. | MOMP gene of |
| | | | avian Chlamydia |
| | | | psittaci types A, C |
| | | | and E”. Unpublished |
| | | | (1994). |
| gi\|129130\|sp\|Q00087\| | **none | 392 aa; xrefs: | Storey, C., |
| | | gi: 40564, gi: | Lusher, M., Yates, P. |
| | | 40565, gi: | and Richmond, S. |
| | | 97245; C. | 1993. MEDLINE |
| | | psittaci. | 94103736. |
| gi\|97245\|pir\|\|A40371\| | **none | strain: | Storey, C., |
| | | FPn/pring; 392 | Lusher, M., Yates, P. |
| | | aa; organism: | and Richmond, S. |
| | | Chlamydophila | 1993. MEDLINE |
| | | psittaci. See | 94103736. |
| | | 129130. | |
| gi\|2935003\|gb\|AAC05028.1\| | gi\|2935002\|gb\|U82958.1\| | >124 aa; omp1; | Kaltenbock, B., |
| | | specific host: | Schmeer, N. and |
| | | Sus scrofa; | Schneider, R. 1997. |
| | | organism: C. suis; | MEDLINE |
| | | submitted | 97339605. |
| | | as C. trachomatis. | |
| gi\|2934999\|gb\|AAC05026.1\| | gi\|2934998\|gb\|U82956.1\| | >124 aa; omp1; | Kaltenbock, B., |
| | | specific host: | Schmeer, N. and |
| | | Sus scrofa; | Schneider, R. 1997. |
| | | organism: C. suis; | MEDLINE |
| | | submitted | 97339605. |
| | | as C. trachomatis. | |
| gi\|2935001\|gb\|AAC05027.1\| | gi\|2935000\|gb\|U82957.1\| | >119 aa; omp1; | Kaltenbock, B., |
| | | specific host: | Schmeer, N. and |
| | | Sus scrofa; | Schneider, R. 1997. |
| | | organism: C. suis; | MEDLINE |
| | | submitted | 97339605. |
| | | as C. trachomatis. | |
| gi\|2935005\|gb\|AAC05029.1\| | gi\|2935004\|gb\|U82959.1\| | >123 aa; omp1; | Kaltenbock, B., |
| | | specific host: | Schmeer, N. and |
| | | Sus scrofa; | Schneider, R. 1997. |
| | | organism: C. suis; | MEDLINE |
| | | submitted | 97339605. |
| | | as C. trachomatis. | |
| gi\|2935007\|gb\|AAC05030.1\| | gi\|2935006\|gb\|U82960.1\| | >127 aa; omp1; | Kaltenbock, B., |
| | | specific host: | Schmeer, N. and |
| | | Sus scrofa; | Schneider, R. 1997. |
| | | organism: C. suis; | MEDLINE |
| | | submitted | 97339605. |
| | | as C. trachomatis. | |
| gi\|2982487\|emb\|CAA06186.1\| | gi\|2982486\|emb\|AJ004877.1\| | 347 aa; omp1; | Hoelzle, L. E., |
| | | specific host: | Steinhausen, G., |
| | | Sus scrofa; | Eggemann, G., |
| | | organism: C. suis; | Schiller, I. and |
| | | submitted | Wittenbrink, M. M. |
| | | as C. trachomatis. | “Isolation and |
| | | | Differentiation of |
| | | | Chlamydia spp. from |
| | | | swine”. |
| | | | Unpublished. |
| gi\|3090413\|emb\|CAA06623.1\| | gi\|3090412\|emb\|AJ005616.1\| | 347 aa; omp1; | Hoelzle, L. E., |
| | | specific host: | Steinhausen, G., |
| | | Sus scrofa; | Eggemann, G., |
| | | organism: C. suis; | Schiller, I. and |
| | | submitted | Wittenbrink, M. M. |
| | | as C. trachomatis. | “Isolation and |
| | | | Differentiation of |
| | | | Chlamydia spp. from |
| | | | swine”. |
| | | | Unpublished. |
| gi\|2982491\|emb\|CAA06188.1\| | gi\|2982490\|emb\|AJ004879.1\| | 347 aa; omp1; | Hoelzle, L. E., |
| | | specific host: | Steinhausen, G., |
| | | Sus scrofa; | Eggemann, G., |
| | | organism: C. suis; | Schiller, I. and |
| | | submitted | Wittenbrink, M. M. |
| | | as C. trachomatis. | “Isolation and |
| | | | Differentiation of |
| | | | Chlamydia spp. from |
| | | | swine”. |
| | | | Unpublished. |
| gi\|5032265\|gb\|AAD38213.1\| | gi\|144570\|gb\|M73043.1\| | 322 aa; omp1; | Kaltenboeck, B., |
| | | specific host: | Kousoulas, K. G. and |
| | | Sus scrofa; | Storz, J. 1993. |
| | | organism: C. suis; | MEDLINE |
| | | submitted | 93123168. |
| | | as C. psittaci. | |
| gi\|2934997\|gb\|AAC05025.1\| | gi\|2934996\|gb\|U82955.1\| | 119 aa; omp1; | Kaltenbock, B., |
| | | Chlamydophila | Schmeer, N. and |
| | | sp. PEENT; | Schneider, R. 1997. |
| | | host: peacock; | MEDLINE |
| | | submitted as | 97339605. |
| | | Chlamydia sp. | |
| gi\|2934995\|gb\|AAC05024.1\| | gi\|2934994\|gb\|U82954.1\| | 139 aa; omp1; | Kaltenbock, B., |
| | | Chlamydiaceae | Schmeer, N. and |
| | | gen. sp. | Schneider, R. 1997. |
| | | EQVAG; host: | MEDLINE |
| | | horse; | 97339605. |
| | | submitted as | |
| | | Chlamydia sp. | |
| gi\|3090407\|emb\|CAA06620.1\| | gi\|3090406\|emb\|AJ005613.1\| | 352 aa; isolate | Hoelzle, L. E., |
| | | pm112; | Steinhausen, G., |
| | | Chlamydophila | Eggemann, G., |
| | | abortus; | Schiller, I. and |
| | | submitted as | Wittenbrink, M. M. |
| | | Chlamydia | “Isolation and |
| | | psittaci. | Differentiation of |
| | | | Chlamydia spp. |
| | | | From swine”. |
| | | | Unpublished. |

| Symbols | Explanation |
|---|---|
| *Protein Accession Number and Nucleotide | Refer to the gi\|gb\|emb\|pir\|sp\|reference numbers |
| Accession Number | as listed. |
| **none | No nucleotide reference was described for this |
| | particular record. |

1. A method for determining the presence or absence of Chlamydia in a patient, comprising the steps of:
(a) obtaining a biological sample from the patient;
(b) contacting at least a portion of the biological sample with a first oligonucleotide that hybridizes to a sequence of a Chlamydia genome under highly stringent conditions;
(c) contacting at least a portion of the biological sample with a second oligonucleotide that hybridizes to a sequence of a Chlamydia cryptic plasmid under highly stringent conditions;
(d) detecting in the sample amounts of a polynucleotide that hybridizes to either the first and second oligonucleotides; and
(e) comparing the amount of polynucleotide that hybridizes to either oligonucleotide to a control value, and therefrom determining the presence of Chlamydia in the patient.

2. The method of claim 1, wherein the first oligonucleotide hybridizes to an ompA sequence.

3. The method of claim 1, wherein the second oligonucleotide hybridizes to an open reading frame 8 sequence of the cryptic plasmid.

4. The method of claim 1, wherein the first oligonucleotide hybridizes to an ompA sequence, and the second oligonucleotide hybridizes to an open reading frame 8 sequence of the cryptic plasmid.

5. The method of claim 1, wherein said Chlamydia is Chlamydia trachomatis.

6. The method of claim 1, wherein said control value is a predetermined cut-off value.

7. The method of claim 1, wherein said control value is a negative control value determined using a third oligonucleotides that does not bind a Chlamydia sequence under high stringency conditions.

8. A plurality of oligonucleotide primers that bind under high stringency conditions to C. trachomatis sequences, wherein two primers bind a genomic sequence, and two primers that bind a cryptic plasmid sequence.

9. The plurality of primers of claim 8, further comprising a probe that binds to a C. trachomatis genomic sequence located between the binding sites of the two primers that bind a genomic sequence.

10. The oligonucleotide primers of claim 8, wherein the primers that bind the genomic sequence are at a physically discrete location from the primers that bind the cryptic plasmid sequence.

11. A diagnostic kit comprising
(a) a first oligonucleotide that hybridizes to a sequence of a Chlamydia genome under highly stringent conditions; and
(b) a second oligonucleotides that hybridizes to a sequence of a Chlamydia cryptic plasmid under highly stringent conditions.

12. The kit of claim 11, wherein the first oligonucleotide hybridizes to an ompA sequence.

13. The kit of claim 11, wherein the second oligonucleotide hybridizes to an open reading frame 8 sequence of the cryptic plasmid.

14. The kit of claim 11, wherein the first oligonucleotide hybridizes to an ompA sequence, and the second oligonucleotide hybridizes to an open reading frame 8 sequence of the cryptic plasmid.

15. The method of claim 11, wherein said Chlamydia is Chlamydia trachomatis.

16. The kit of claim 11, further comprising a positive control polynucleotide sequence that binds to either the first or second oligonucleotide under high stringency conditions.

17. The kit of claim 11, further comprising a negative control polynucleotide sequence that does not bind to either the first or second oligonucleotides under high stringency conditions.

18. The kit of claim 11, wherein one or more of the oligonucleotides are fluorescently labeled.


SEQUENCE LISTING (8 sequences)

SEQ ID NO: 1 - DNA, 23 bp, Artificial Sequence
  Probe or primer to detect ompA sequences
atgaaaaaactcttgaaatcggt

SEQ ID NO: 2 - DNA, 20 bp, Artificial Sequence
  Probe or primer to detect ompA sequences
cgatcataaggcttggttca

SEQ ID NO: 3 - DNA, 12 bp, Artificial Sequence
  Probe or primer to detect ompA sequences
  modified_base (6)...(6): I
  misc_feature 6: n = A,T,C or G
ttagtntttgcc

SEQ ID NO: 4 - DNA, 17 bp, Artificial Sequence
  Probe or primer to detect ompA sequences
  modified_base 6: I
  misc_feature 6: n = A,T,C or G
ttagtntttgccgcttt

SEQ ID NO: 5 - DNA, 25 bp, Artificial Sequence
  Probe or primer to detect cryptic plasmid sequences
aaaagagtttcaatcgatcccctat

SEQ ID NO: 6 - DNA, 25 bp, Artificial Sequence
  Probe or primer to detect cryptic plasmid sequences
gagttcgacattccacatactttcc

SEQ ID NO: 7 - DNA, 30 bp, Artificial Sequence
  Probe or primer to detect cryptic plasmid sequences
aggcgatttaaaaaccaaggtcgatgtgat

SEQ ID NO: 8 - DNA, 30 bp, Artificial Sequence
  Probe or primer to detect cryptic plasmid sequences
tcacatcatcgaccttggtttttaaatcgc